(19) United States
(12) Patent Application Publication
(10) Pub. No.: US 2006/0287229 A1
(43) Pub. Date: Dec. 21, 2006

(54) Novel CD40 variants

(76) Inventors: Dani Eshel, Pardes-Hana Karkur (IL); Galit Rotman, Herzliya (IL); Kinneret Savitsky, Tel-Aviv (IL); Amir Toporik, Azur (IL); Rami Khosravi, Herzilia (IL); Aviva Chen, Ramat Gan (IL); Yona Bismuth, Tel-Aviv (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/172,493

(22) Filed: Jun. 30, 2005

Related U.S. Application Data
(60) Provisional application No. 60/584,153, filed on Jul. 1, 2004.

(51) Int. Cl. A61K 38/17

(52) U.S. Cl. 514/12

(57) ABSTRACT

The invention concerns CD40 skipping 5 nucleic acid sequences and amino acid sequences obtained by alternative splicing of CD40, pharmaceutical compositions comprising said sequences and methods for treatment of a disease, wherein a beneficial therapeutic effect is achieved by the up regulation of the CD40-R-CD40-L interaction. An antibody capable of selectively binding to the amino acid of CD40 skipping 5 and pharmaceutical composition comprising the above antibody and methods for detecting the presence of exon 5 skipping expression in a sample are also within the scope of the invention.

### RELATED APPLICATIONS

This application claims priority to U.S. Ser. No. 60/584,153, filed Jul. 1, 2004. The contents of this application are incorporated herein by reference in their entirety.

### FIELD OF THE INVENTION

The present invention relates to pharmaceutical compositions comprising a soluble variant of CD40 or comprising antibodies reactive with amino acid sequences of the soluble variant of CD40, and methods of use and of treatment thereof.

### BACKGROUND OF THE INVENTION

CD40 was originally described as a receptor responsible for the activation and differentiation of B-lymphocytes. This receptor engages to its ligand (CD154, also named “CD40-L”; CD40 receptor is sometimes referred to as “CD40-R”), promoting cell survival and costimulatory protein expression necessary for interaction with T-lymphocytes. Thus, interaction of B- and T-cells via the CD40-CD154 system allows mutual activation, with B-cells secreting antibodies and T-cells becoming effector cells producing cytokines.

However, the CD40-CD154 system has wider implications than just activation of B- and T-lymphocytes. CD40 is also expressed by migratory immune cells, such as macrophages and dendritic cells, which present antigens and activate T-lymphocytes. Engagement of CD40 by T-lymphocyte CD154 activates these immune cells to express new immune modulators, such as the cytokines IL-1, IL-12 and TNF. Additionally, non-hematopoietic cells, including fibroblasts, endothelial cells, smooth muscle cells and some epithelial cells, constitutively display CD40 on their surface, and that this expression is upregulated following exposure to IFN. CD40 signaling in non-hematopoietic cells via CD154 results in initiation of cellular functions, such as synthesis of pro-inflammatory cytokines. CD40 engagement on endothelial and vascular smooth muscle cells induces synthesis of matrix matalloproteinases (MMP), which degrades collagens and other connective tissue proteins crucial for the stability of atherosclerotic plaques and their fibrous caps.

Initially, it was thought that CD154 is expressed only on the surface of T-lymphocytes after their activation. However, CD154 was also found to be expressed by eosinophils and mast. In addition, human platelets have pre-formed CD154 inside them. Once activated by thrombin or other mediators, platelet internal stores of CD154 are exported to the surface where some is secreted. Several other cell types are now known to have CD154 stored within. These include macrophages, B-lymphocytes, endothelial cells and smooth muscle cells.

A number of pathological processes of chronic inflammatory diseases in humans, and several experimental animal models of chronic inflammation, were shown to be dependent upon or involve the CD40-CD154 system (Xu Y, Song G., J Biomed Sci. 2004 July-August;11(4):426-38.; Chitnis T, Khoury S J., J Allergy Clin Immunol. 2003 November;112(5):837-49; Flavell R A. Curr Top Microbiol Immunol. 2002;266:1-9). These include graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, as well as hematological malignancies and other cancers (Tong A W, Stone M J. Cancer Gene Ther. 2003 January;10(1):1-13; Flavell R A. Curr Top Microbiol Immunol. 2002;266:1-9). A remarkable spectrum of chronic inflammatory conditions can be blocked or substantially reduced by disrupting the CD40-CD154 system. These studies typically employ either mice with targeted disruption of either CD40 or CD154 genes, or use neutralizing monoclonal anti-CD154 antibodies. These antibodies appear to work by disrupting the communication bridge constructed by CD40-CD154. The animals in these experimental models appear to be no worse for having this system disrupted for months.

Targeting CD40-CD154 signaling, either by blocking these interactions or by stimulating the signaling, was shown to be therapeutically beneficial. At least two different companies are testing anti-human CD154 antibodies for efficacy in diseases such as systemic lupus erythematosus, graft-versus-host disease, and tissue transplantation. Trials are ongoing with much promise for success.

A critical role for CD40-CD154 has been established for several autoimmune diseases, including lupus nephritis, systemic lupus erythematosus, rheumatoid arthritis, diabetes mellitus and multiple sclerosis. Treatment of such diseases by blocking the costimulatory pathway involving CD40-CD154 are currently being tested (Kyburz D, Carson D A, Corr M., Arthritis Rheum. 2000 November;43(11):2571-7; Kelsoe G., J Clin Invest. 2003 November;112(10):1480-2; Huang W X, Huang P, Hillert J. Mult Scler. 2000 April;6(2):61-5). Studies using several animal models of autoimmune diseases show that disease symptoms can be blocked or substantially reduced by disrupting the CD40-CD154 system. Recently it was found that agonistic anti-CD40 antibodies can also reduce progression and severity of a murine model for rheumatoid arthritis, suggest that activating agents of this pathway may also be used in therapy of pathological cases of chronic inflammation.

Treatment of autoimmune diseases, including lupus nephritis, systemic lupus erythematosus, rheumatoid arthritis, diabetes mellitus and multiple sclerosis by agonistic CD40 antibodies was described in PCT application WO 01/37870, hereby incorporated by reference as if fully set forth herein. This application discloses methods of treating autoimmune diseases comprising administering an agonistic anti-CD40 antibody. The application demonstrates that agonistic anti-CD40 mAb have a remarkable therapeutic effect on blocking and/or ameliorating the development of arthritis, in a model for CCIA (Chronic Collagen Induced Arthritis), thus indicating their potential clinical use to control chronic inflammatory conditions of autoimmune origin.

The involvement of CD40-CD154 in lupus, nephritis and SLE has been extensively investigated. Several models of murine lupus have been used to investigate the potential therapeutic efficacy of interrupting the CD40-CD154 system, and all have shown impressive inhibition of autoantibody production and nephritis, and improved survival. Concurrent therapy with anti-CD154 antibodies and CTLA4-Ig showed dramatic synergistic effects that lasted long after treatment was discontinued. Particularly encouraging are the findings that treated mice were shown to maintain the capacity to mount an effective immune response after completion of therapy.

Phase I clinical trials with anti-CD154 antibodies were carried out in patients with SLE (Kalunian K C., et al. Arritis Rheum. 2002 December;46(12):3251-8). These studies indicated that the agent was well tolerated. However, in another study, thromboembolic complications were reported, possibly due to the particular antibody that was used (Koyama I, et al., Transplantation. 2004 Feb. 15;77(3):460-2; Kawai T, et al., Nat Med. 2000 February;6(2):114). Some anti-CD40 antibodies are known to be stimulatory, for example, acting as agonists rather than antagonists. Thus, the precise nature of the antibody being used would be expected to result in the varying appearance of many effects related to CD40-CD154 interactions, in addition to unexpected effects that are not related to these interactions. The synovial tissue in RA patients is emiched with mature antigen presenting cells (APCs) and many lymphocytes. Interactions and signaling through the costimulatory CD40-CD154 and CD28-CD80/86 molecules are involved in the initiation and amplification of the inflammatory reactions in the synovium. Thus, blocking such signaling pathways might provide a specific immunotherapeutic approach for the treatment of RA. Indeed, prevention of collagen-induced arthritis (CIA), a murine model for RA, was observed upon administration of anti-CD154 antibody. Treatment with anti-CD154 also prevented arthritis development in a model of immunoglobulin-mediated arthritis.

The CD40-CD154 system plays a critical role in the response of the immune system to an invading pathogen, leading to an antigen-driven lymphoproliferative process. When downregulation of this tightly controlled mechanism is impaired, lymphoproliferative disorders may occur. CD40 expression is elevated in malignant B- and T-cell lymphomas, and in Reed-Sternberg cells of Hodgkin's disease. CD154 is constitutively expressed in several types of B-cell lymphoid malignancies. Furthermore, approximately 50% of patients with these malignancies have elevated levels of biologically active soluble CD154 in their serum. The effect of CD40 activation in B-cell malignancies has been examined extensively by use of activating anti-CD40 antibodies or soluble CD154. Whenever primary human malignant B cells were analyzed, CD40 activation consistently enhanced malignant cell survival and mediated their resistance to chemotherapy (Ottaiano A, et al, Tumori. 2002 September-October;88(5):361-6; Fiumara P, Younes A., Br J Haematol. 2001 May;113(2):265-74; Kipps T J, Chu P, Wierda W G. Semin Oncol. 2000 December;27(6 Suppl 12): 104-9; Szocinski J L., et al. Blood. 2002 Jul. 1;100(1):217-23).

Taken together, the co-expression of CD40 and CD154 by malignant B cells, the presence of soluble CD154 in the sera of these patients, and the ability of CD40 activation to enhance malignant B-cell survival, suggest that CD40/CD154 may provide an autocrine/paracrine survival loop for malignant B cells. Thus, interrupting CD40/CD154 interaction may be of therapeutic value in patients with B-cell lymphoid malignancies. Anti-CD154, but surprisingly also stimulatory antibodies to CD40, were successfully tested as immunotherapy for malignant B cell tumors in murine models.

Elevated expression of CD40 was described in other forms of cancer, including epithelial neoplasia, nasopharyngeal carcinoma, osteosarcoma, neuroblastoma and bladder carcinoma. Recombinant soluble CD154 inhibited the growth of CD40(+) human breast cell lines in vitro, due to increased apoptosis. In addition, treatment of tumor-bearing mice with this molecule resulted in increased survival.

New chimeric or fully human monoclonal antibodies (or its antigen-binding portion thereof) were developed that specifically bind to and activate human CD40, as described in PCT application WO03/040170, hereby incorporated by reference as if fully set forth herein. These antibodies were shown to be useful for treating cancer, HIV, neutropenia or viral infections, by enhancing the human immune response. CD40 activation by anti-CD40 antibody was shown to eradicate CD40+ and CD40− lymphoma in mouse models (French R. R. et al., Nature Medicine 1999, 5:548-53).

Furthermore, studies by Glennie and co-workers conclude that signaling activity by anti-CD40 antibodies is more effective for inducing in vivo tumor clearance than other anti-surface marker antibodies capable of recruiting effectors (Tutt A. L. et al., J of Immunol. 1998, 161:3176-85). In another study, bone marrow dendritic cells (DCs) were treated ex vivo with a variety of agents, and tested for in vivo antitumor activity. These studies demonstrated that CD40L stimulated DCs were the most mature and most effective cells that mounting an antitumor response. The essential role of CD40 in antitumor immunity has also been demonstrated by comparing responses of wild-type and CD40−/− mice to tumor vaccines. These studies show that CD40−/− mice are incapable of achieving the tumor immunity observed in normal mice. (Mackey M. F. et al., Cancer Research 1997, 57:2569-74). In another study, splenocytes from tumor bearing mice were stimulated with tumor cells and treated with activating anti-CD40 antibodies ex vivo, and were shown to have enhanced tumor specific CTL activity. (Donepudi M. et al., Cancer ImmunoL Immunother, 1999, 48:153-164). These studies demonstrate that CD40 occupies a critical position in antitumor immunity, in both CD40 positive and negative tumors. Another aspect of CD40/CD154 in the treatment of malignancies is the potential use of CD154 in immune gene therapy, since CD40/CD154 interaction has been shown to be critical for generating protective T cell-mediated anti-tumor response (Tong A W, Stone M J., Cancer Gene Ther. 2003 January;10(1):1-13; Kipps T J, Int J Hematol. 2002 August;76 Suppl 1:269-73). In this approach, CD154 is transferred ex-vivo into neoplastic cells, by infection with a modified adenovirus. The results of a Phase I study in CLL patients show induction of autologous cytotoxic T cells capable of destroying the neoplastic B cells, concomitant with significant reduction in leukemic cell counts and lymph node sizes. Furthermore, this approach appears to enhance antibody-dependent cellular cytotoxicity, and thereby augment the activity of antitumor monoclonal antibody therapy. Thus, this approach alone or in combination with tumor-specific Mab therapy (such as Rituxan, anti-CD20), may offer an effective strategy for the treatment of B-cell malignancies. Transduction of tumor cells ex vivo with CD154, in solid tumors such as neuroblastoma and squamous cell carcinoma, can induce immune responses against the tumor cells, mediating rejection or impeding tumor growth.

Activating CD40 signaling was also shown to reduce bone cell death or apoptosis associated with osteoporosis, osteonecrosis and inflammatory arthritis. CD40 is expressed on bone cells and CD40 agonists were shown to dramatically reduce bone cell death. US Patent Application No. US20030099644, hereby incorporated by reference as if fully set forth herein, discloses methods and uses of agonistic anti-CD40 antibodies to reduce or prevent bone cell death or apoptosis, thereby providing new treatments for bone loss associated with a variety of diseases and clinical conditions. The bone cells that can be treated by CD40 agonists include, but are not limited to, osteoblasts and osteocytes. The CD40 agonists inhibit the apoptosis of osteocytes and/or osteoblasts to a greater extent than osteoclasts, and otherwise produce a net beneficial effect on bone mass, bone density, bone cell number or other parameter indicative of health bone tissue. Agents that “stimulate” cell signaling via CD40 receptors may do so directly or indirectly. Although agents that act directly are generally preferred, agents that indirectly stimulate or activate CD40 receptors may be used, including accessory signaling molecules, co-stimulators and the like, and agents that remove, inactivate or downregulate inhibitors of the CD40 signaling process. Included within this group of CD40 agonists are agents that stimulate or “upregulate” the expression of the CD40 receptor on bone cells.

Activated T-lymphocytes not only express cell membrane-associated but also soluble CD154. The kinetics of soluble CD154 (sCD154) expression resemble expression patterns observed for the membrane-associated form, though the mechanisms of generation and/or release of sCD154 remain poorly understood. Several studies suggest that sCD154 retains the ability to interact with CD40. Recently, the soluble forms of CD154 have received more attention, particularly in association with certain human diseases. Enhanced levels of sCD154 have been detected in patients with disorders such as active SLE, unstable angina, and B-Cell lymphoma (Komura K et al, J Rheumatol. 2004 March;31(3):514-9; Conde I D, Kleiman N S., N Engl J Med. 2003 Jun. 19;348(25):2575-7; author reply 2575-7., Heeschen C, et al, N Engl J Med. 2003 Mar. 20;348(12): 1104-11).

Soluble CD40 (sCD40) was detected in culture supernatants from CD40-positive cell lines, but not from CD40-negative cells. A substantial proportion of sCD40 in these cultures retained ligand binding activity. High levels of sCD40 were also observed in supernatants from AIDS-related lymphoma B-cell lines. sCD40 that was expressed by B cells was shown to bind CD154 on activated T cells, and is thought to regulate CD40-CD154 in a negative fashion. sCD40 was also detected in serum and urine of healthy donors, and was highly elevated in patients with impaired renal function, including chronic renal failure, haemodialysis and chronic ambulatory peritoneal dialysis (CAPD) patients (Contin C, Immunology. 2003 September;110(1):131-40.). Patients with neoplastic disease and chronic inflammatory bowel disease (CIBD) (Schwabe, R. F., et al, Clin. Exp. Immunol, 1999, 117:153-158) showed slight but significant elevations of sCD40 in their serum.

A recent study suggested that sCD40 can be created through alternative splicing (Tone, M., et al., 2001, PNAS 98:1751-1756). As such, sCD40 molecules may have unique antigenic epitopes, distinct from CD40, which could be used to raise sCD40-specific antibodies.

At least one study suggests that expression of sCD40 regulates CD40-CD154 interactions in a positive fashion. Given the ample evidence for a critical role of CD40-CD154 in injury, inflammation and cancer, it appears that targeting this system may prove to play an important therapeutic role in abating inflammation in a variety of diseases and in cancer treatment. Reports that agonistic anti-CD40 antibodies can also reduce severity of disease and disease progression suggest that activating this pathway may be useful for therapy of pathological cases of chronic inflammation.

Monoclonal antibody targeting of the CD40-CD154 pathway has shown beneficial effects in a number of experimental animal models. However, whether these techniques can be applied to humans remains to be determined, since treatment with humanized antibodies has obvious limitations. Other options for modulating this pathway with higher specificity and efficacy, such as sCD40, hold promise as therapeutic agents.

Splice variants of the transcript that encodes CD40 have been isolated, characterized and cloned. These splice variants include naturally occurring sequences obtained by alternative splicing of the known known CD40 gene depicted as CD40 HUMAN Swiss Prot. under Accession Number P25942, SEQ ID NO:3 for protein and SEQ ID NO:4 for nucleic acid sequence, which is incorporated herein by reference. These splice variants are not merely truncated forms, or fragments of the known gene, but rather novel sequences that naturally occur within the body of individuals. Different splice variants encoded by a single gene may be expressed in vivo in different physiological situations and may result in activation of distinct cellular pathways. These splice variants include nucleic acid molecules that encode the extracellular region of CD40 or a fragment thereof, linked to a unique tail sequence. The extracellular region may be fully conserved, or there may be deletions, insertions or substitutions. In some variants the translation product of the splice variant is a soluble protein that retains the CD40 function of binding to CD40 ligands such as CD154 or CD40 itself.

U.S. Pat. No. 6,720,182, by the inventors, hereby incorporated by reference as if fully set forth herein, discloses the sequences of a large number of splice variants. Among others this application also discloses a CD40 splice variant lacking exon 6 (“skipping 6”) and having in addition a unique tail at amino acids 166-203. CD40-skipping 6 variant was described also in the PNAS paper (Tone, M., et al., 2001, PNAS 98:1751-1756).

WO 01/05967 by the inventors, hereby incorporated by reference as if fully set forth herein, discloses a splice variant of CD40, termed “skipping 5”, which contains three out of the four extracellular exons of the known CD40 gene, but does not contain exon 5. Skipping 5 additionally contains a unique sequence present as amino acids 135-160, which is not present in the known CD40 MRNA transcript.

WO03/070768 by the inventors, hereby incorporated by reference as if fully set forth herein, discloses three CD40 secreted splice variants, termed NJ1, NJ2, NJ3, each of which contains all four of the extracellular exons, in addition to at least one unique taile sequence not present in the known CD40 mRNA transcript. U.S. Ser. No. 10/979,178 by the inventors, hereby incorporated by reference as if fully set forth herein, discloses three additional CD40 secreted splice variants, VAR1, VAR2 and VAR3. The splice variant in U.S. Pat. No. 6,720,182 lacking exon 6, of WO 01/05967 lacking exon 5, the three splice variants of WO03/070768 and the three splice variants of 10/979,178, all lack the transmembrane domain of the receptor and are thus secreted.

All publications, patents and patent applications mentioned in this specification are herein incorporated in their entirety by reference into the specification, to the same extent as if each individual publication, patent or patent application was specifically and individually indicated to be incorporated herein by reference.

### SUMMARY OF THE INVENTION

The present invention is based on the finding that the protein transcribed from a specific CD40 splice variant, termed “skipping 5”, has unique pharmaceutical and biochemical properties, and has agonistic effects with regard to the CD40/CD154 system. The skipping 5 variant has been shown to result in an increase in physiological activities associated with interactions of known CD40 with CD154.

As demonstrated in Table 1, the CD40 skipping 5 variant is a soluble CD40 variant, lacking the transmembrane domain of the wild type CD40, having a unique tail which spans amino acids 136-160 of the variant sequence. As demonstrated in Table 2, the CD40 skipping 5 variant has all the amino acid residues required for CD154 ligand binding.

The activity of CD40 skipping 5 variant was demonstrated using an in-vitro model that involves the induction of cytokine RANTES secretion by human mesothelial cells upon their ligation with CD154 expressing cells. Typical soluble CD40 proteins are expected to compete with the membrane-bound receptor for binding to the CD40 ligand and to reduce the CD40 receptor/CD40 ligand interaction, thereby leading for example to a reduced secretion of cytokines such as RANTES. Unexpectedly, the CD40 skipping 5 splice variant according to the invention has an opposite effect: it increases cytokine production. In other words, CD40 skipping 5 variant acts as an agonist. As described in greater detail below, when the skipping 5 protein was administered to a mixture of human peritoneal cells and mouse fibroblasts transfected to express the CD154 ligand, skipping 5 was able to raise the level of secretion of the cytokine RANTES, as compared to when an interferon control was administered alone, or as compared to when other soluble variants of cd40, such as the skipping 6 variant or the truncated extracellular portion of the known CD40 (both of which lack the unique sequence of the skipping 5 variant, which spans amino acids 136-160 of the variant sequence), were administered. RANTES is a cytokine whose secretion is indicative of T cell activation. In addition, the skipping 5 mRNA transcript has been found to have a physiological expression pattern which is different from that of known CD40. Namely, the level of the skipping 5 transcript rises when apoptosis is induced in erythroleukemic cells, while the level of known CD 40 decreases when apoptosis is induced. Diseases in which apoptosis is involved can be divided into two groups: those in which there is an increase in cell survival (ie diseases associated with inhibition of apoptosis), and those in which there is an increase in cell death (and hence hyperactive apoptosis). There are many studies demonstrating that cell apoptosis plays a relevant role in the etiology of many diseases. Furthermore, many different pharmacologic agents (cytotoxic agents, hormones, anti-inflammatory drugs) incur their effects through induction of apoptosis of target cells. (Ramirez et al. 1999. Apoptosis and disease. Alergol. Immunol. Clin. 6: 367-374).

Thus, the skipping 5 protein could be useful when administered as a pharmaceutical composition for up regulation and activation of the naturally occurring CD40 receptor/CD40 ligand (CD40/CD154) interaction.

Up regulation of CD40 receptor activities may be beneficial, for example, for treating diseases in which it is desired to increase the activity of the immune system, such as for treating cancer for example, and/or for treating diseases characterized by a lack of activation of the immune system, and/or for treating diseases in patients who suffer from a weakened or less functional immune system, such as elderly patients or patients suffering from HIV/AIDS for example.

Up regulation of CD40 receptor activities may be also beneficial for treating tumors, such as lymphomas, leukemias, multiple myeloma, carcinomas of nasopharynx, bladder, ovary and liver, breast and colorectal cancers.

Up regulation of CD40 receptor activities may be further beneficial for treating autoimmune diseases, such as rheumatoid arthritis, systemic lupus erythematosis, diabetes myellitis or multiple sclerosis.

Up regulation of CD40 receptor activities may be further beneficial to reduce bone cell death or apoptosis associated with osteoporosis, osteonecrosis and inflammatory arthritis. The therapeutic benefits of the upregulation of CD40 are described in more detail in the Background section of the specification.

The skipping 5 protein is encoded by the nucleotide sequence shown in SEQ ID NO:2, shown in the attached sequence listing. The availability of the naturally occurring protein of the present invention, as an alternative to activation of the naturally occurring CD40 receptor/CD40 ligand interaction with a synthetic agent and/or antibody, provides therapeutic alternatives to those patients who do not respond to CD40 receptor activating agents, or as an alternative to CD40 receptor activating agents which can cause substantial adverse side effects.

In another embodiment, the present invention relates to bridges, tails, and/or insertions, and/or analogs, homologs and derivatives of such peptides. Such bridges, tails, and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding “known protein”, while at least a second portion of the variant comprises the tail.

As used herein “an edge portion” refers to a connection between two portions of a splice variant according to the present invention that were not joined in the known CD40 proteins. An edge may optionally arise due to a join between the above “known protein” portion of a variant and the tail, for example, and/or may occur if an internal portion of the known CD40 sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

As used herein the phrase “known protein” refers to a known CD40 or other database provided sequence of a specific protein, including, but not limited to, SwissProt (http://ca.expasy.org/), National Center of Biotechnology Information (NCBI)(http://www.ncbi.nlm.nih.gov/), PIR (http://pir.georgetown.edu/), A Database of Human Unidentified Gene-Encoded Large Proteins [HUGE <http://www.kazusa.orjp/huge>], Nuclear Protein Database [NPDhttp://npd.hgu.mrc.ac.uk], human mitochondrial protein database (http://bioinfo.nist.gov:8080/examples/servlets/index.html), and University Protein Resource (UniProt) (http://www.expasy.uniprot.org/).

In one embodiment, an isolated chimeric polypeptide encoding for CD40 skipping 5, comprising a first amino acid sequence being at least about 90%, preferably at least about 95% homologous to amino acids 1-135 corresponding to the known CD40 sequence (SEQ ID NO:3) and a second amino acid sequence being at least about 70%, optionally at least 80%, preferably at least 85%, more preferably at least 90% and most preferably at least 95% homologous to a polypeptide having the sequence VRPKTWLCNRQAQTRLMLSVVPRIG, wherein said first and said second amino acid sequences are contiguous and in a sequential order.

In another embodiment, an isolated polypeptide chimeric encoding for a tail of CD40 skipping 5, comprising a polypeptide having the sequence being at least about 70%, optionally at least 80%, preferably at least 85%, more preferably at least 90% and most preferably at least 95% homologous to a polypeptide having the sequence VRPKTWLCNRQAQTRLMLSVVPRIG.

In another embodiment, an isolated chimeric polypeptide encoding for an edge portion of CD40 skipping 5 corresponding to SEQ ID NO: 1, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AV having a structure as follows (numbering according to SEQ ID NO: 1): a sequence starting from any of amino acid numbers 135−x to 135 and ending at any of amino acid numbers 136+((n−2)−x), in which x varies from 0 to n−2, such that the value ((n−2)−x) is not allowed to be larger than 24.

For example, for peptides of 10 amino acids (such that n=10), the starting position could be as “early” in the sequence as amino acid number 127 if x=n−2=8 (ie 127=135−8), such that the peptide would end at amino acid number 136 (136+(8−8=0)). On the other hand, the peptide could start at amino acid number 135 if x=0 (ie 135=135−0), and could end at amino acid 144 (144=136+(10−2)−0).

According to other embodiments, the bridge portion above, comprising a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: QIAV, IAVR, AVRP. All peptides feature AV as a portion thereof. Peptides of from about five to about nine amino acids could optionally be similarly constructed.

The present invention further provides, a pharmaceutical composition comprising a pharmaceutically acceptable carrier, and as an active ingredient an agent comprising an amino acid sequence selected:
i. the amino acid sequence depicted in SEQ ID No: 1;
ii. a fragment of at least 10 amino acids the amino acid sequence of (i), having at least four consecutive amino acids of the segment 136-160 of SEQ ID No: 1, as depicted in SEQ ID NO:5, said fragment still having CD40-L binding properties substantially as those of the sequence of (i);
iii. a variant of the amino acid sequence of (i) or (ii) wherein up to 20% of the amino acids have been replaced, chemically modified or deleted, wherein the sequence substantially maintains the CD40-L binding properties of (i);
iv. a chimeric protein comprising the amino acid sequence of (i), (ii) or (iii), conjugated to another entity;
v. an isolated chimeric polypeptide encoding for CD40 skipping 5, comprising a first amino acid sequence being at least about 90% homologous, preferably at least about 95% to amino acids 1-135 corresponding to known CD40 and a second amino acid sequence being at least about 70%, optionally at least 80%, preferably at least 85%, more preferably at least 90% and most preferably at least 95% homologous to a polypeptide having the sequence VRPKTWLCNRQAQTRLMLSVVPRIG, wherein said first and said second amino acid sequences are contiguous and in a sequential order;
vi. an isolated chimeric polypeptide encoding for a tail of CD40 skipping 5, comprising a polypeptide having the sequence being at least about 70%, optionally at least 80%, preferably at least 85%, more preferably at least 90% and most preferably at least 95% homologous to a polypeptide having the sequence VRPKTWLCNRQAQTRLMLSVVPRIG;
vii. an isolated chimeric polypeptide encoding for an edge portion of CD40 skipping 5 corresponding to SEQ ID NO: 1, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AV having a structure as follows (numbering according to SEQ ID NO: 1): a sequence starting from any of amino acid numbers 135−x to 135 and ending at any of amino acid numbers 136+((n−2)−x), in which x varies from 0 to n−2, such that the value ((n−2)−x) is not allowed to be larger than 24.

The phrase “substantially maintains CD40-L binding properties” means the protein, e.g., a CD40-skipping 5 protein, variant or fragment thereof, binds specifically to a CD40-L. In some embodiments, the protein binds to a CD40L molecule at a concentration at which binding of wild-type CD40 is not detected.

Table 1 below shows a comparison of CD40 skipping 5 variant with other known splice variants of CD40 regarding various parameters. Table 1, as well as the above described characteristics of CD40 skipping 5 variant underscore the fact that CD40 skipping 5 variant is not merely a truncated form of the gene but rather naturally occurring splice variant. Table 2 below summarizes the presence of amino acids known to be involved in the ligand binding in various CD40 variants. All the numbers of amino acid residues in Tables 1 and 2 below are according to the WT residues (SEQ ID NO:3).

In Table 1, domains and pattern were analyzed according to INTERPRO, which is a database of protein families, domains and functional sites, including identified features. These features, which are known to occur in particular (previously identified) proteins, can be applied to unknown protein sequences by using this data. The tool can be found at: www.ebi.ac.uk/interpro/. Further description of Interpro can be found in Mulder et al., (2003) Nucleic Acids Res. 31, 315-318. INTERPRO was used to deduce the C6 domain, which is a TNFR/NGFR domain and the TNFR2 domain.

In Table 1, disulfide bonds are given according to Swiss-Prot Swiss-Prot is a well known protein database, which includes information about disulfide bonds for known proteins (see Bairoch et al., (2004) Brief. Bioinform. 5:39-55). In this case, the information about these bonds was taken from the database for the wild type (WT) protein. The variant proteins were then checked to see if they had the same residues, and hence form similar disulfide bonds. Identification of disulfide bonds was based on TNFR (tumor necrosis factor receptor) as CD40 protein is also called “Tumor necrosis factor receptor superfamily member 5.” Swiss-Prot database was also used to identity of the signal peptide (SP) and predict the domains of the CD40 proteins (based on SwissProt accession number P25942 of the known CD40 (WT)).

In Table 1, glycosylation sites were identified using ProScan, which is software that performs a ProSite Scan. ProSite is a database which can be used to identify protein features and also related proteins to a sequence. See Hulo et al., (2004) Nucl. Acids. Res. 32:D134-D137.

GRAVY is a hydrophobicity parameter (See Kyte & Doolittle, (1982) J Mol Biol 157:105-132). TMpred relates to predictions of transmembrane domains.

TABLE 1
CD40 alternatively spliced variant proteins

| | WT | Skip6¹ | Skip5 | NJ1² | NJ2² | NJ3² | VAR1³ | VAR2³ | VAR3³ |
|---|---|---|---|---|---|---|---|---|---|
| Amino acid No. | 277 | 203 | 160 | 244 | 191 | 237 | 166 | 151 | 248 |
| Amino acid overlap | 277 | 1-165 | 1-134 | 1-187 | 1-187 | 1-187 | 1-135 | 1-134 | 1-186 |
| with WT | | | | | | | | | 215-277 |
| Molecular weight | 30618 | 22259 | 18032 | 26723 | 21045 | 25769 | 18677 | 17035 | 27418 |
| (Da), (+sp) | | | | | | | | | |
| Molecular weight | 28258 | 19898 | 15671 | 24362 | 18684 | 23408 | 16316.4 | 14674 | 25057 |
| (Da), (−sp) | | | | | | | | | |
| Theoretical pI | 5.49 | 5.39 | 6.29 | 5.58 | 5.01 | 5.12 | 6.05 | 5.42 | 5.19 |
| (+sp) | | | | | | | | | |
| Theoretical pI | 5.39 | 5.28 | 6.23 | 5.48 | 4.9 | 5.02 | 5.96 | 5.26 | 5.09 |
| (−sp) | | | | | | | | | |
| GRAVY (+sp) | −0.267 | −0.428 | −0.323 | −0.352 | −0.245 | −0.419 | −0.343 | −0.366 | −0.533 |
| GRAVY (−sp) | −0.369 | −0.590 | −0.519 | −0.476 | −0.395 | −0.554 | −0.533 | −0.582 | −0.672 |
| TMpred | +++ | − | − | − | − | − | − | − | − |
| C6 domain | 4 | 3 | 2/3 | 4 | 4 | 4 | 2/3 | 2/3 | 4 |
| Disulfide bonds | | all | 125-143 | all | all | all | 125-143 | 125-143 | all |
| | | | missing | | | | missing | missing | |
| EGF_2 103-116 | + | + | + | + | + | + | + | + | + |
| WTN-linked | ++ | +− | −− | ++ | ++ | ++ | −− | −− | ++ |
| Glycosylation | | | | | | | | | |
| (153-6,180-3) | | | | | | | | | |

TABLE 2
Presense of amino acids involved in the ligand binding

| | WT | Skip6¹ | Skip5 | NJ1² | NJ2² | NJ3² | VAR1³ | VAR2³ | VAR3³ |
|---|---|---|---|---|---|---|---|---|---|
| Ligand binding | + | + | + | + | + | + | + | + | + |
| amino acids: E74 | | | | | | | | | |
| Y82 N86 D84 E114 | | | | | | | | | |
| E117 | | | | | | | | | |

¹The term “skip6” refer to the CD40 skipping exon 6 variant, disclosed in U.S. Pat. No. 6,720,182 and in U.S. Patent Application No. 09/569611, by the inventors, hereby incorporated by reference as if fully set forth herein.
²The terms “NJ1”, “NJ2” and “NJ3” refer to CD40 splice variants described in PCT Application WO03/070768, by the inventors, hereby incorporated by reference as if fully set forth herein.
³The terms “VAR1”, “VAR2” and “VAR3” refer to CD40 splice variants described in US patent application 10/979,178, by the inventors, hereby incorporated by reference as if fully set forth herein.

In the following the term “skipping 5” refers in general to any one of the sequences (i)-(vii) above, all of which are characterized in having at least part of the unique tail of amino acids 136-160, at SEQ ID NO: 1, and are clearly differentiated in comparison to soluble CD40 and other splice variants of CD40 in that they lack the exon of the extracellular domain.

Without wishing to be limited by a single hypothesis, the ability of the CD40 splice variant to bind CD154 may be due to the presence of particular amino acids as follows: positions E74, Y82, N86, D84, E114, E117 of CD40, in both the known CD40 and variant sequences. According to certain embodiments of the invention, the fragment of (ii) above preferably comprises one or more regions containing these amino acids critical for the activity of CD154 binding domains, preferably linked to each other (either in the order appearing in the native protein or in another order) optionally through the use of spacers, and further linked to said at least four consecutive amino acids of the CD40 skipping exon 5 variant sequence from the tail portion (amino acids 136-160 of SEQ ID NO: 1). The amino acids at positions E74, Y82, N86, D84, E114, E117 of the CD40 are preferably either maintained as in the parent (known CD40) sequence, or substituted by conservative substitution.

The segment “136-160” of SEQ ID NO:1, as depicted in SEQ ID NO: 5, is the unique tail of the CD40 splice variant skipping 5, a segment which does not appear in the known CD40 sequence or in any other of the known splice variants.

Four consecutive amino acids may be any four amino acids of this tail such as, for example, 136-139, 137-140, 138-141 . . . 157-160.

The consecutive amino acids may be five (136-140, 137-141 . . . 156-160), six (136-141 . . . 155-160), seven, eight or nine.

Preferably at least 10 consecutive amino acids of the unique segment 136-160, and most preferably all the amino acids of this unique segment should be present in the fragment.

The term “up to 20%” means that at least 80% of the variant sequence are identical to those of (i) or (ii), so that a combination of no more than 20 has been deleted, and/or replaced and/or chemically modified.

Moreover, in one embodiment of the invention, up to 15% of the amino acids have been replaced, chemically modified or deleted (i.e. 85% are identical with (i) or (ii), wherein the sequence maintains the CD40-L binding properties of (i). In another embodiment, up to 10% of the amino acids have been replaced, chemically modified or deleted (i.e. 90% are identical with (i) or (ii), wherein the sequence maintains the CD40-L binding properties of (i). In a third embodiment, up to 5% of the amino acids have been replaced, chemically modified or deleted (i.e. 95% are identical with (i) or (ii), wherein the sequence maintains the CD40-L binding properties of (i).

Additionally, in certain embodiments of the invention, in the chimeric protein of (iv), (v), (vi) or (vii), the amino acid sequence of (i), (ii) or (iii), is conjugated to an entity selected from a member of the following group: an antibody or an antibody fragment, preferably an F_{c} fragment, a glycoprotein, a fragment of the comp protein, b-zip (Morris A. E., et al, JBC, 274: 418-423, 1999) In such case, in some embodiments the antibody fragment originates in the F_{c} region of an antibody of IgG1.

Further, in some embodiments, in (i), (ii), (iii), (iv), (v), (vi) or (vii) up to 20% of the amino acid of the native sequence has been replaced with a naturally or non-naturally occurring amino acid or with a peptidomimetic organic moiety; and/or up to 20% of the amino acids have their side chains chemically modified and/or up to 20% of the amino acids have been deleted, provided that at least 80% of the amino acids in the parent sequence of (i), (ii), (iii), (iv), (v), (vi) or (vii) are maintained unaltered, and provided that the amino acid maintains the biological activity of the parent sequence of (i), (ii), (iii), (iv), (v), (vi) or (vii).

Still further, in some embodiments, in (i), (ii), (iii), (iv), (v), (vi) or (vii) at least one of the amino acids is replaced by the corresponding D-amino acid, which replacement increases the protein's resistance to degradation by naturally present enzymes.

Additionally, in certain embodiments, in (i), (ii), (iii), (iv), (v), (vi) or (vii) the peptidic backbone of at least one of the amino acids has been altered to a non-naturally occurring peptidic backbone, which replacement increases the protein's resistance to degradation by naturally present enzymes.

The present invention further provides a method for treatment of a disease, wherein a beneficial therapeutic effect is achieved by the modification of the CD40-R-CD40-L interaction, comprising administering to an individual in need of such treatment, a therapeutically effective amount of the composition of the present invention. This composition is comprised of, a pharmaceutically acceptable carrier, and as an active ingredient an agent comprising an amino acid sequence selected from:
i. the amino acid sequence depicted in SEQ ID No: 1;
ii. a fragment of at least 10 amino acids the amino acid sequence of (i), having at least four consecutive amino acids of the segment 136-160 of SEQ ID No:1, as depicted in SEQ ID NO:5, said fragment still having CD40-L binding properties substantially as those of the sequence of (i);
iii. a variant of the amino acid sequence of (i) or (ii) wherein up to 20% of the amino acids have been replaced, chemically modified or deleted, wherein the sequence substantially maintains the CD40-L binding properties of (i);
iv. a chimeric protein comprising the amino acid sequence of (i), (ii) or (iii), conjugated to another entity;
v. an isolated chimeric polypeptide encoding for CD40 skipping 5, comprising a first amino acid sequence being at least about 90%, preferably at least about 95% homologous to amino acids 1-135 corresponding to the known CD40 sequence SEQ ID NO: 3 and a second amino acid sequence being at least about 70%, optionally at least 80%, preferably at least 85%, more preferably at least 90% and most preferably at least 95% homologous to a polypeptide having the sequence VRPKTWLCNRQAQTRLMLSVVPRIG, wherein said first and said second amino acid sequences are contiguous and in a sequential order;
vi. an isolated chimeric polypeptide encoding for a tail of CD40 skipping 5, comprising a polypeptide having the sequence VRPKTWLCNRQAQTRLMLSVVPRIG;
vii. an isolated chimeric polypeptide encoding for an edge portion of CD40 skipping 5 corresponding to SEQ ID NO: 1, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AV having a structure as follows (numbering according to SEQ ID NO: 1): a sequence starting from any of amino acid numbers 135−x to 135 and ending at any of amino acid numbers 136+((n−2)−x), in which x varies from 0 to n−2, such that the value ((n−2)−x) is not allowed to be larger than 24.

Preferably, the disease is selected from hematological malignancies and other cancers (including but not limited to human leukemias, lymphomas, and multiple myeloma, epithelial neoplasia, nasopharyngeal carcinoma, osteosarcoma, neuroblastoma and bladder carcinoma, ovary and liver carcinomas, breast and colorectal cancers, AIDS-related lymphoma), impaired renal function, including chronic renal failure, and for treatment of patients requiring haemodialysis and chronic ambulatory peritoneal dialysis (CAPD).

Preferably, the disease is selected from clinical conditions associated with bone loss, including but not limited to osteoporosis, osteonecrosis and inflammatory arthritis.

Preferably, the disease is selected from autoimmune diseases such as: lupus nephritis, systemic lupus erythematosus, rheumatoid arthritis, multiple sclerosis, inflammatory bowel diseases (IBD), ulcerative colitis, Crohn's disease, hematological malignancies, Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Lupus (SLE), Grave's disease, myasthenia gravis, autoimmune hemolytic anemia, autoimmune thrombocytopenia, and asthma.

Further, the present invention relates to use of a sequence as defined in (i)-(vii) above, for preparing a medicament for the treatment of a disease, wherein a beneficial therapeutic effect is achieved by the up regulation of the CD40-R biological activities. The use may optionally be for any of the above applications, and/or as a diagnostic marker and/or antibody target.

Thus the present invention concerns a pharmaceutical composition comprising a pharmaceutically acceptable carrier and as an active ingredient an antibody capable of selectively binding to an epitope found in SEQ ID NO: 1, while essentially not binding to wild-type soluble CD40.

As used herein the term “unique tail” is meant to refer to the amino acid sequence at the C terminus of the CD40 splice variant, which sequence does not appear in the known CD40. The unique tail region (SEQ ID NO:5) of the CD40 splice variant SEQ ID NO: 1 spans amino acids 136-160 of SEQ ID NO: 1.

In the present invention, the term “ligand” or “CD40 ligand” or “CD40-L” is meant to refer not only to CD154, but to any other compounds such as TRAF3 or TRAF2 which are known to interact with CD40.

In the present invention, the term “CD40-R-CD40-L interaction” refers to the interaction between the CD40 receptor and at least one of its ligands.

As used herein, the term “fragments” as applied to protein fragments of the CD40 splice variant refers to those fragments which are at least 10 amino acids long which include at least 4 consecutive amino acids of the unique tail region (defined as amino acids 136-160 in SEQ ID NO: 1). In some preferred embodiments the fragments includes 5, 6, 7, 8, 9, preferably above 10, most preferably all the amino acids of the unique tail region. In some preferred embodiments of SEQ ID NO: 1, the fragment includes 10, 11, 12, 13, 14, 15, 16, or 17 amino acids of the unique tail region.

It should be noted that in accordance with the invention several fragments which in the native skipping 5 variant of SEQ ID NO: 1 are spaced apart may be limited to each other in tandem either directly or through suitable spaces.

According to other embodiments, an isolated nucleic acid molecule encoding a CD40 skipping exon 5 variant having a nucleic acid sequence as set forth in any one of SEQ ID NO:2 or homologs thereof.

Reference is further made to the nucleic acid sequence of SEQ ID No 2, which is an exemplary nucleic acid sequence coding for SEQ ID No. 1, which may be used in the production of SEQ ID No: 1, and/or may be used as a probe and/or to design such a probe in the detection of expression of the protein in a sample.

According to other embodiments, an expression vector comprising the polynucleotide sequence encoding a CD40 skipping exon 5 variant having a nucleic acid sequence as set forth in any one of SEQ ID NO:2 or homologs thereof.

According to other embodiments, a host cell comprising the vector comprising the polynucleotide sequence encoding a CD40 skipping exon 5 variant having a nucleic acid sequence as set forth in any one of SEQ ID NO:2 or homologs thereof.

As used herein the term “nucleic acid sequence” is meant to refer to a sequence composed of DNA nucleotides, RNA nucleotides or a combination of both types and may include natural nucleotides, chemically modified nucleotides and synthetic nucleotides.

The term “amino acid” refers either to one of the 20 naturally occurring amino acids to a peptidomimetic (see below), or to a D or L residue having the following formula:
—NH—CHR—CO—

wherein R is an aliphatic group, a substituted aliphatic group, a benzyl group, a substituted benzyl group, an aromatic group or a substituted aromatic group and wherein R does not correspond to the side chain of a naturally occurring amino acid. This term also refers to the D-amino acid counterpart of naturally occurring amino acids. Amino acid analogs are well known in the art; a large number of these analogs are commercially available. Many times the use of non-naturally occurring amino acids in the peptide has the advantage that the peptide is more resistant to degradation by enzymes which fail to recognize them.

As used herein the term “variants” is meant to refer to amino acid sequences in which one or more (up to 20 amino acids) has been added, deleted or replaced as compared to the parent sequence or the parent fragment.

As used herein the term “substitution” refers both to conservative and non conservative substitutions.

The term “conservative substitution” in the context of the present invention refers to the replacement of an amino acid present in the native sequence, with a naturally or non-naturally occurring amino or a peptidomimetics (see below) having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

To produce conservative substitutions by non-naturally occurring amino acids it is also possible to use amino acid analogs (synthetic amino acids) well known in the art. A peptidomimetic of the naturally occurring amino acid is well documented in the literature and known to the skilled practitioner.

When affecting conservative substitutions the substituting amino acid should have the same or a similar functional group in the side chain as the original amino acid.

The following are some non-limiting examples of groups of naturally occurring amino acids or of amino acid analogs. Replacement of one member in the group by another member of the group will be considered herein as a conservative substitution:

Group I includes: leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably Group I includes leucine, isoleucine, valine and methionine.

Group II includes: glycine, alanine, valine, serine, cysteine, threonine and a modified amino acid having an ethyl side chain. Preferably Group II includes glycine and alanine.

Group III includes: phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes: glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO-NH-alkylated glutamine or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes: histidine, lysine, arginine, N-nitroarginine, β-cycloarginine, μ-hydroxyarginine, N-amidinocitruline and 2-amino-4-guanidinobutanoic acid, homologs of lysine, homologs of arginine and omithine. Preferably, Group V includes histidine, lysine, arginine, and ornithine. A homolog of an amino acid includes from 1 to about 3 additional methylene units in the side chain.

Group VI includes: serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amine yielding lysine-epsilon amino cysteine or alanine-beta amino cysteine, respectively.

The term “non-conservative substitutions” concerns replacement of the amino acid as present in the native skipping 5 protein by another naturally or non-naturally occurring amino acid, having different electrochemical and/or steric properties, for example as determined by the fact the replacing amino acid is not in the same group as the replaced amino acid of the native protein sequence. Those non-conservative substitutions which fall under the scope of the present invention are those which still constitute a compound having CD40 agonist activity as described herein. Because D-amino acids have hydrogen at a position identical to the glycine hydrogen side chain, D-amino acids or their analogs can often be substituted for glycine residues, and are a preferred non-conservative substitution

A “non-conservative substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid.

Alternatively, a functional group may be added to the side chain, deleted from the side chain or exchanged with another functional group. Examples of non-conservative substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄ COOH for the side chain of serine. These examples are not meant to be limiting.

As used herein the term “chemically modified” when referring to a protein of the invention, is meant to refer to a protein where at least one of its amino acid residues is modified either by natural processes, such as processing or other post-translational modifications, or by chemical modification techniques which are well known in the art. Among the numerous known modifications typical, but not exclusive examples, include: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

As used herein the term “having at least 80% identity” with respect to two amino acid or nucleic acid sequence sequences, is meant to refer to the percentage of residues that are identical in the two sequences when the sequences are optimally aligned. Thus, 80% amino acid sequence identity means that 80% of the amino acids in two or more optimally aligned polypeptide sequences are identical.

As used herein the term “deletion” is meant to refer to the absence of one or more amino acids which may be at terminal or non terminal regions and which absence may be of several consecutive or non consecutive amino acid residues.

As used herein the terms “insertion” and “addition” is meant to refer to that change in a nucleotide or amino acid sequence which has resulted in the addition of one or more nucleotides or amino acid residues, respectively, as compared to the naturally occurring sequence.

As used herein the term “substitution” is meant to refer to replacement of one or more amino acids by different amino acids. As regards amino acid sequences the substitution may be conservative or non-conservative.

As used herein the term “alternative splicing” is meant to refer to exon exclusion, deletion of terminal or non terminal sequences in the variants as compared to the original sequence, as well as to intron inclusion of sequences originally not appearing in the parent sequence.

As used herein, the term “effective amount” refers to an amount of active ingredient which is an ingredient comprising any of the sequences (i) to (iv) or in order to prevent, ameliorate or cure a disease or postpone deterioration of a disease and is determined by such considerations as may be known in the art. The amount must be effective to achieve the desired therapeutic effect by administering the amino acid sequences of the invention, to a person in need thereof. The amount depends, inter alia, on the type and severity of the disease to be treated and the treatment regime. The effective amount is typically determined in appropriately designed clinical trials (dose range studies) and the person versed in the art will know how to properly conduct such trials in order to determine the effective amount.

According to preferred embodiments of the present invention, preferably any of the nucleic acid and/or amino acid sequences featured herein further comprises any sequence having at least about 70%, preferably at least about 80%, more preferably at least about 90%, most preferably at least about 95% homology thereto.

All nucleic acid sequences and/or amino acid sequences shown herein as embodiments of the present invention relate to their isolated form, as isolated polynucleotides (including for all transcripts), oligonucleotides (including for all segments, amplicons and primers), peptides (including for all specific regions described hereni, optionally including other antibody epitopes as described herein) and/or polypeptides (including for all proteins). It should be noted that oligonucleotide and polynucleotide, or peptide and polypeptide, may optionally be used interchangeably.

According to other embodiments, the invention provides an antibody specifically recognizing the isolated CD40 skipping exon 5 variant and polypeptide fragments of this invention. Preferably such an antibody differentially recognizes CD40 skipping exon 5 variant of the present invention but do not recognize known CD40 peptides.

Optionally amino acid sequence corresponds to a tail as described herein. Also optionally, the antibody is capable of differentiating between a splice variant having the epitope and a corresponding known protein, such as known CD40 proteins described herein.

According to preferred embodiments of the present invention, there is provided at least one primer pair capable of selectively hybridizing to a nucleic acid sequence as described herein. According to other preferred embodiments, there is provided at least one oligonucleotide capable of selectively hybridizing to a nucleic acid sequence as described herein.

According to preferred embodiments of the present invention, there is provided a nucleic acid construct comprising the isolated polynucleotide as described herein.

Optionally, the nucleic acid construct further comprises a promoter for regulating transcription of the isolated polynucleotide in sense or antisense orientation.

Optionally, the nucleic acid construct further comprises a positive and a negative selection marker for selecting for homologous recombination events.

According to preferred embodiments of the present invention, there is provided a host cell comprising the nucleic acid construct as described herein.

According to preferred embodiments of the present invention, there is provided an isolated polypeptide comprising an amino acid sequence at least 70% identical to a polypeptide as described herein, as determined using the LALIGN software of EMBnet Switzerland (http://www.ch.embnet.org/index.html) using default parameters or an active portion thereof.

According to preferred embodiments of the present invention, there is provided an oligonucleotide specifically hybridizable with a nucleic acid sequence encoding a polypeptide as described herein.

According to preferred embodiments of the present invention, there is provided a pharmaceutical composition comprising a therapeutically effective amount of a polypeptide as described herein and a pharmaceutically acceptable carrier or diluent.

According to preferred embodiments of the present invention, there is provided a method of treating CD40-related disease in a subject, the method comprising upregulating in the subject expression of a polypeptide as described herein, thereby treating the CD40-related disease in a subject. Optionally, upregulating expression of said polypeptide is effected by:

(i) administering said polypeptide to the subject; and/or

(ii) administering an expressible polynucleotide encoding said polypeptide to the subject.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

### BRIEF DESCRIPTION OF THE DRAWINGS

FIG. 1: presents the pTen21 plasmid map and multiple cloning site sequences.

FIG. 2: schematic presentation of the pTen21 vector digested with EcoRV and BglII and ligated to the amplified PCR fragment of CD40wtEC, digested with the same enzymes.

FIG. 3: schematic presentation of the pTen21-CD40 wtEC clone 7 vector digested with StuI and BglII and ligated to the amplified PCR fragment of CD40-skipping 5, digested with the same enzymes.

FIGS. 4a and 4b: show the full length sequence of the Vector pTen21. The primers (SEQ ID NOS:11 and 12) are marked in bold and underlined.

FIGS. 4c and 4d: show the Vector pTen21-CD40 wtEC (EC refers to the extracellular domain of CD40 WT) full length sequence. The primers are marked in bold and underlined, in bold italic is the CD40 wtEC coding sequence. Also shown are the BamHI-EcoRV and BglII sites. The—signal peptide-encoding sequence is presented in the rectangle.

FIGS. 5a and 5b: shows the Vector pTen21-CD40_-Skipping 5 full length sequence. The primers are marked in bold and underlined, in bold italic is the CD40_Skipping 5 coding sequence. Also shown are BamHI-EcoRV and BglII sites.

FIG. 6: presents the sequence determined for Clone 7 as described in SEQ ID NO:9, featuring the BamHI-EcoRV and BgmI sites marked in bold, encompassing the CD40wtEC sequence.

FIG. 7: presents the sequence determined for Clone 8 as described in SEQ ID NO:14, featuring the BamHI-EcoRV and BglII sites marked in bold, encompassing the CD40_Skipping 5 sequence, which is shown in bold italic.

FIGS. 8a and 8b: show the Vector pTen21-Fc full length sequence. The OQBT primers are marked in bold and underlined, the Fc sequence is colored in bold italics. Also shown is the polylinker

FIG. 9: shows the Fc sequence within the pTen21-Fc vector, as shown in SEQ ID NO: 16, featuring the XhoI and KpnI sites marked in bold and encompassing the Fc sequence, which is also shown in bold.

FIG. 10: schematic presentation of the pTen21-Fc clone 19 digested with EcoRV and BglII and ligated to the amplified PCR fragment of CD40wtEC, digested with the same enzymes.

FIG. 11a and 11b: show the Vector pTen21-cd40wtEC-Fc full length sequence. The primers are marked in bold and underlined. The CD40wtEC-Fc fusion sequences are shown in bold italics, and are separated by the tacgta sequence.

FIG. 12: presents the sequence determined for clone 37, as presented in SEQ ID NO: 19, featuring the BamHI-EcoRV and KpnI sites marked in bold. The CD40 wtEC-Fc fusion sequences are shown in bold italics, and are separated by the tacgta sequence.

FIG. 13: schematic presentation of the pTen21-CD40wtEC-Fc clone 37 vector digested with StuI and BglII and ligated to the amplified PCR fragment of CD40-skipping 5, digested with the same enzymes.

FIGS. 14a and 14b: show the Vector pTen21-CD40_Skipping 5-Fc vector, presented in SEQ ID NO:21, where the primers are marked in bold and underlined, the CD40-skipping 5-Fc fusion sequences fusion sequences are shown in bold italics, and are separated by the tacgta sequence. Also shown are the polylinker and the signal peptide-encoding sequence, which is shown with a rectangle.

FIG. 15: presents the sequence determined for clone 9, as presented in SEQ ID NO:22, where the internal StuI, BamHI and BglII sites are underlined. The BamHI and EcoRV sites upstream of the ATG are in Bold. The BamHI and EcoRV sites upstream of the ATG are in Bold. The CD40-skipping 5 fusion sequences are shown in bold italics, and are separated by the tacgta sequence.

FIG. 16: Western blot analysis of the purified CD40 proteins as follows: lane 1 presents CD-40wtEC-Fc protein, lane 2 presents CD-40wt-Fc protein, lane 3 presents CD-40 skipping 6-Fc protein, and lane 4 presents the TNFRII-Fc negative control, recognised by a commercially available polyclonal antibody N-16 (polyclonal rabbit antibody from Santa Cruz (Cat num. Sc-974)).

FIG. 17: shows the results of FACS analysis, demonstrating sCD40 binding to CD154 ligand. Detailed description of the experiments is provided in Example 6, in Examples section below. FIG. 17A: represents the results of the Fc-tagged CD40-skipping 6 variant binding to mouse fibroblasts, stably transfected with full length human CD154. FIG. 17B: represents the results of the Fc-tagged CD40-skipping 5 variant binding to mouse fibroblasts, stably transfected with full length human CD154. FIG. 17C: represents the results of the Fc-tagged CD40-WT binding to mouse fibroblasts, stably transfected with full length human CD154. FIG. 17D: represents the negative control, as the mouse fibroblasts used do not express CD154 ligand. FIG. 17E: summarizes the mean fluorescence shift, as plotted versus various concentrations of CD40 protein.

FIGS. 18A-G: demonstrate the Effect of the CD40 variant on RANATES secretion. The ability of the soluble CD 40 skipping 5 protein to compete with the CD40 membrane-bound receptor for binding to the secreted CD154 ligand was tested in these experiments as compared to soluble wild-type CD40 protein and to the CD 40 skipping 6 protein. Detailed description of the experiments is provided in Example 7, in the Example section below. FIG. 18A: represents the results of the control situation, where the HPMC cells were untransfected and thus did not express CD154 ligand. FIG. 18B: represents the results of the control situation, where the mouse fibroblasts used in conjunction with the HPMC cells were untransfected and thus did not express CD154 ligand. FIG. 18C: represents the results of the experiment, where the mouse fibroblasts used in conjunction with the HPMC cells were transfected to express CD154 ligand. INF and an appropriate commercially available anti-CD40 antibody were used as positive controls. The concentration of the administered CD40 proteins is indicated. FIG. 18D:shows the results of titration of stimulated RANTES production by varying CD154+ mouse fibroblasts number. FIG. 18E:presents the contrasting effects of stimulated RANTES production by WT soluble CD40, shown in pink, and the CD40 skipping 5 variant, shown in blue. FIG. 18F: represents the control experiment for dose response assay, with mouse fibroblasts that do not express the CD154 ligand. FIG. 18G: represents the dose dependent inhibition of RANTES secretion by soluble CD40 proteins with mouse fibroblasts stably expressing the CD154 ligand.

FIG. 19: demonstrates the RT-PCR results showing the mRNA expression of the CD40 variants in K652 cells.

FIG. 20: demonstrates the RT-PCR results showing the alteration of the expression pattern of the different variants of CD40 as a response to apoptosis in K562 cells.

FIG. 21: presents the percentage of the expression of each splice form out of the total CD40 expression levels in K562 cells treated with 20 μM Etoposide for various time intervals.

FIG. 22: demonstrates immunoblotting results of K562 cells treated with 25 μM etoposide for 17 hours to induce apoptosis, in order to measure the activation of caspases. The cells were lysed and immunobloted, and the PARP substrate for caspase-3 was probed using anti cleaved PARP antibody.

### DETAILED DESCRIPTION OF THE INVENTION

The present invention is based in part on the finding that the protein transcribed from a specific CD40 splice variant, termed “CD40 skipping exon 5” or “skipping 5”, has unique pharmaceutical and biochemical properties, and has agonistic effects with regard to the CD40/CD154 system. The skipping 5 variant has been shown to result in an increase in physiological activities associated with interactions of known CD40 with CD154.

Agonistic CD40 receptor activities may be beneficial, for example, for treating diseases in which it is desired to increase the activity of the immune system, such as for treating cancer for example, and/or for treating diseases characterized by a lack of activation of the immune system, and/or for treating diseases in patients who suffer from a weakened or less functional immune system, such as elderly patients or patients suffering from HIV/AIDS for example.

Agonistic CD40 receptor activities may be also beneficial for treating tumors, such as lymphomas, leukemias, multiple myeloma, carcinomas of nasopharynx, bladder, ovary and liver, breast and colorectal cancers.

Agonistic CD40 receptor activities may be further beneficial for treating autoimmune diseases, such as rheumatoid arthritis, systemic lupus erythematosis, diabetes myellitis or multiple sclerosis.

Agonistic CD40 receptor activities may be further beneficial to reduce bone cell death or apoptosis associated with osteoporosis, osteonecrosis and inflammatory arthritis. The therapeutic benefits of the upregulation of CD40 are described in more detail in the Background section of the specification.

Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, 0, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×106 cpm 32P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×106 cpm 32P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; (ii) hybridization solution of 6×SSC and 0.1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include 3H, 14C, 32P, and 35S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Optionally and preferably, nucleic acid sequence homology (identity) is determined using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C-NH, CH2-O, CH2-CH2, S═C-NH, CH═CH or CF═CH, backbone modifications, and residue modification. Methods for preparing peptidomimetic compounds are well known in the art and are specified. Further details in this respect are provided hereinunder.

Peptide bonds (—CO-NH—) within the peptide may be substituted, for example, by N-methylated bonds (—N(CH3)-CO—), ester bonds (—C(R)H-C-O-O-C(R)-N—), ketomethylen bonds (—CO-CH2—), *-aza bonds (—NH-N(R)-CO—), wherein R is any alkyl, e.g., methyl, carba bonds (—CH2-NH—), hydroxyethylene bonds (—CH(OH)-CH2—), thioamide bonds (—CS-NH—), olefinic double bonds (—CH═CH—), retro amide bonds (—NH-CO—), peptide derivatives (—N(R)-CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

TABLE 3

| Non-conventional amino acid | Code |
|---|---|
| α-aminobutyric acid | Abu |
| α-amino-α-methylbutyrate | Mgabu |
| aminocyclopropane-Carboxylate | Cpro |
| aminoisobutyric acid | Aib |
| aminonorbornyl-Carboxylate | Norb |
| Cyclohexylalanine | Chexa |
| Cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-α-methylalnine | Dnmala |
| D-α-methylarginine | Dnmarg |
| D-α-methylasparagine | Dnmasn |
| D-α-methylasparatate | Dnmasp |
| D-α-methylcysteine | Dnmcys |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | mser |
| L-α-methylvaline | Mtrp |
| L-α-methylleucine | Mval Nnbhm |
| N-(N-(2,2-diphenylethyl)carbamylmethyl-glycine | Nnbhm |
| 1-carboxy-1-(2,2-diphenylethylamino)cyclopropane | Nmbc |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgin |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcyclopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cyclododeclglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylserine | Dnmser |
| D-N-methylthreonine | Dnmthr |
| N-(1-methylethyl)glycine | Nva |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| Penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| N-(3-guanidinopropyl)glycine | Narg |
| N-(1-hydroxyethyl)glycine | Nthr |
| N-(hydroxyethyl)glycine | Nser |
| N-(imidazolylethyl)glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| Penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl)carbamylmethyl(1)glycine | Nnbhe |

Since the peptides of the present invention are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

Antibodies

According to other embodiments, there is provided an antibody specifically recognizing CD40 skipping exon 5 variant of the present invention. The antibody or antibody fragment comprises an immunoglobulin specifically recognizing CD40 skipping exon 5 variant or a portion thereof. The term “specifically recognizing” when referring to an antibody, refers to a binding reaction that is determinative of the presence of the protein in a heterogeneous population of proteins. Thus, under designated immunoassay conditions, the specified antibodies bind to a particular protein at least about two times the background and do not substantially bind in a significant amount to other proteins present in the sample. Thus, preferably such an antibody differentially recognizes CD40 skipping exon 5 variant of the present invention but does not recognize known CD40 peptides, such as wild type CD40 protein (SEQ ID NO:3), CD40 skipping exon 6 (SEQ ID NO:24), described in U.S. Pat. No. 6,720,182, by the inventors; CD40 variants NJ1, NJ2, NJ3 (SEQ ID NOs: 25, 26, 27, respectively), described in WO03/070768 by the inventors, CD40 variants VAR1, VAR2 and VAR3 (SEQ ID NOs: 28, 29, 30, respectively), described in U.S. patent application Ser. No. 10/979,178, by the inventors, all hereby incorporated by reference as if fully set forth hereinAccording to still other embodiments, the antibody or antibody fragment specifically recognizes an amino acid sequence corresponding to or homologous to a CD40 skipping exon 5 variant according to the present invention, as shown for example by SEQ ID NO: 1, or a fragment thereof comprising at least one CD40 skipping exon 5 variant variant epitope. The term “epitope” refers to a site on an antigen to which B and/or T cells respond. B-cell epitopes can be formed both from contiguous amino acids or noncontiguous amino acids juxtaposed by tertiary folding of a protein. As used herein, the term “epitope” further relates to epitopes useful to distinguish between the Splice Variant of this invention and known peptides.

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [whitlow and Filpula, Methods 2: 97-105 (1991); Bird et al., Science 242:423-426 (1988); Pack et al., Bio/Technology 11: 1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intem. Rev. Immunol. 13, 65-93 (1995).

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

Upregulating Methods and Agents

It will be appreciated that the present methodology for treatment may be effected by specifically upregulating the expression of the variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-5R gene, which include (i.e., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the known CD40 receptor and increase the expression of the shorter isoforms. Design and synthesis of oligonucleotides which can be used according to the present invention are described hereinbelow and by Sazani and Kole (2003) Progress in Molecular and Subcellular Biology 31:217-239.

Alternatively or additionally, upregulation may be effected by administering to the subject at least one polypeptide agent of the polypeptides of the present invention or an active portion thereof, as described hereinabove. However, since the bioavailability of large polypeptides is relatively small due to high degradation rate and low penetration rate, administration of polypeptides is preferably confined to small peptide fragments (e.g., about 100 amino acids).

An agent capable of upregulating a CD40 skipping exon 5 variant polypeptide may also be any compound which is capable of increasing the transcription and/or translation of an endogenous DNA or MRNA encoding the CD40 skipping exon 5 variant polypeptide and thus increasing endogenous CD40 activity.

An agent capable of upregulating a CD40 skipping exon 5 variant may also be an exogenous polypeptide including at least a functional portion (as described hereinabove) of the CD40.

Upregulation of CD40 skipping exon 5 variant can be also achieved by introducing at least one CD40 substrate. Non-limiting examples of such agents include HOXC10 (Gabellini D, et al., 2003; EMBO J. 22: 3715-24), human securin and cyclin B1 (Tang Z, et al., 2001; Mol. Biol. Cell. 12: 3839-51), cyclins A, geminin H, and Cut2p (Bastians H, et al., 1999; Mol. Biol. Cell. 10: 3927-3941).

It will be appreciated that upregulation of CD40 skipping exon 5 variant can be also effected by administration of CD40 skipping exon 5 variant-expressing cells into the individual.

CD40 skipping exon 5 variant-expressing cells can be any suitable cells, such as lung, ovary, bone marrow which are derived from the individual and are transfected ex vivo with an expression vector containing the polynucleotide designed to express CD40 skipping exon 5 variant as described hereinabove.

Administration of the CD40 skipping exon 5 variant-expressing cells of the present invention can be effected using any suitable route such as intravenous, intra peritoneal, and intra muscular, for example. According to presently preferred embodiments, the CD40 skipping exon 5 variant-expressing cells of the present invention are introduced to the individual using intravenous and/or intra organ administrations.

CD40 skipping exon 5 variant-expressing cells of the present invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Downregulating Methods and Agents

Downregulation of CD40 skipping exon 5 variant can be effected on the genomic and/or the transcript level using a variety of molecules which interfere with transcription and/or translation (e.g., antisense, siRNA, Ribozyme, DNAzyme), or on the protein level using e.g., antagonists, enzymes that cleave the polypeptide and the like.

Following is a list of agents capable of downregulating expression level and/or activity of CD40 skipping exon 5 variant.

One example, of an agent capable of downregulating a CD40 skipping exon 5 variant polypeptide is an antibody or antibody fragment capable of specifically binding CD40 skipping exon 5 variant. Preferably, the antibody specifically binds at least one epitope of a CD40 skipping exon 5 variant as described hereinabove.

An agent capable of downregulating a CD40 skipping exon 5 variant transcript is a small interfering RNA (siRNA) molecule. RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the CD40 skipping exon 5 variant transcript mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl, T. 2001, ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating a CD40 skipping exon 5 variant transcript is a DNAzyme molecule capable of specifically cleaving an mRNA transcript or DNA sequence of the CD40 skipping exon 5 variant. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther. www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of a CD40 skipping exon 5 variant transcript can also be effected by using an antisense polynucleotide capable of specifically hybridizing with an mRNA transcript encoding the CD40 skipping exon 5 variant.

Design of antisense molecules which can be used to efficiently downregulate a CD40 skipping exon 5 variant must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target MRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a CD40 skipping exon 5 variant transcript is a ribozyme molecule capable of specifically cleaving an MRNA transcript encoding a CD40 skipping exon 5 variant. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Another agent capable of downregulating CD40 skipping exon 5 variant would be any molecule which binds to and/or cleaves CD40 skipping exon 5 variant. Such molecules can be CD40 antagonists, or CD40 inhibitory peptide.

It will be appreciated that a non-functional analogue of at least a catalytic or binding portion of CD40 can be also used as an agent which downregulates CD40 skipping exon 5 variant.

Another agent which can be used along with the present invention to downregulate CD40 skipping exon 5 variant is a molecule which prevents CD40 activation or substrate binding.

Active Ingredient of the Pharmaceutical Composition

The active ingredient agent of the present invention as described herein may be an agent comprising the full sequences of SEQ ID NO: 1, fragment of at least 10 amino acids of SEQ ID NO: 1 which contains at least four consecutive amino acids of the unique tail (SEQ ID NO:5), sequences in which one or more of the amino acid residues in SEQ ID NO: 1 is substituted with a conserved or non-conserved amino acid residue (preferably a conserved amino acid residue); or (ii) sequences in which one or more of the amino acid residues includes a constituent group (chemically modified), or (iii) sequences in which the “skipping 5” CD40 protein or peptide is fused with another compound, such as the F_{c} fragment of an antibody or a compound that increases the half-life of the protein (for example, polyethylene glycol (PEG)), or a moiety which serves as targeting means to direct the protein to its target tissue (such as an antibody or a fragment), or (iv) sequences in which additional amino acids are fused to the “skipping 5” CD40 protein or peptide. Such fragments, variants and derivatives are deemed to be within the scope of the invention for those skilled in the art from the teachings herein.

Substantially purified skipping 5 protein or peptide can be isolated from natural sources, produced by recombinant DNA methods or synthesized by standard protein synthesis techniques. Substantially purified functionally active fragments of skipping 5 protein that comprise at least 10 amino acid residues including 4 amino acid residues of the unique tail sequence can be produced by processing proteins isolated from natural sources, or by recombinant DNA methods or synthesized by standard protein synthesis techniques.

Without wishing to be limited by a single hypothesis, it is believed that skipping 5 proteins or peptides are capable of binding to CD40 ligands (for example CD154), although it is possible that such proteins or peptides could bind to CD40 itself (additionally or alternatively) and could also exert an effect through such binding. In any case, as shown in greater detail below, the physiological effect of the skipping 5 variant protein according to the present invention is to increase or potentiate the physiological effect(s) of CD40. Thus, the skipping 5 proteins or peptides of the present invention may act as “agonists” of CD40.

Skipping 5 proteins or peptides of the invention are soluble and may therefore be administered to a subject for treatment. As “agonists” such treatment would be expected to increase CD40 activities, which are associated with the immune system. For example, skipping 5 proteins could increase signaling activity which occurs when CD40+ cells interact with CD154+ cells (ie cells expressing CD40 and cells expressing CD154, respectively). The soluble alternatively spliced CD40 of the present invention (skipping 5 proteins or peptides) is thus expected to modulate immune activity as a CD40 agonist.

Accordingly, skipping 5 proteins or peptides may optionally be used as an active ingredient in a pharmaceutical composition used to modulate immune activity, particularly for increasing the immune activity or activities associated with CD40-CD154 interactions.

The Active Ingredient of the Invention

In one embodiment of the pharmaceutical composition of the present invention, up to 20% of the amino acids of the native sequence have been replaced with a naturally or non-naturally occurring amino acid or with a peptidomimetic organic moiety; and/or up to 20% of the amino acids have their side chains chemically modified and/or up to 20% of the amino acids have been deleted, provided that at least 80% of the amino acids in the parent sequence is maintained unaltered, and provided that the amino acid maintains the biological activity of the parent sequence.

It should be noted that the active ingredient of the present invention comprises the sequences of (i)-(iv) above. The active ingredient may have also an additional moiety/moieties attached to the C- and/or N-terminal, added for various purposes not related to the agonistic effect on CD40.

The composition may also comprise non-amino acid moieties, such as for example, hydrophobic moieties (various linear, branched, cyclic, polycyclic or hetrocyclic hydrocarbons and hydrocarbon derivatives) attached to the peptides of the skipping 5 variant, to improve penetration through membranes (for delivery purposes). In addition, various protecting groups may be included, which are attached to the compound's terminals to decrease degradation, especially when the peptide is linear. Chemical (non-amino acid) groups may be included in order to improve various physiological properties such as penetration through membranes (moieties which enhance penetration through membranes or barriers); decreased degradation or clearance; decreased repulsion by various cellular pumps, improved immunogenic activities, improved various modes of administration (such as attachment of various sequences which allow penetration through various barriers such as BBB, through the gut, etc.); increased specificity, increased affinity, decreased toxicity, for imaging purposes and the like. The chemical groups may serve as various spacers, placed for example, between one or more of the above binding domains, so as to spatially position them in suitable orientation in respect of each other and in respect of the ligand.

The active ingredient of the invention may be linear or cyclic, and cyclization may take place by any means known in the art. Where the composition is composed predominantly of amino acids/amino acid sequences, cyclization may be N- to C-terminus, N-terminus to side chain and N-terminus to backbone, C-terminus to side chain, C-terminus to backbone, side chain to backbone and side chain to side chain, as well as backbone to backbone cyclization. Cyclization of the compound may also take place through the non-amino acid organic moieties.

The association between the amino acid sequence component of the composition and other components of the composition may be by covalent linking, or by non-covalent complexion, for example, by complexion to a hydrophobic polymer, which can be degraded or cleaved producing a composition capable of sustained release; by entrapping the amino acid part of the composition in liposomes or micelles to produce the final composition of the invention. The association may be by the entrapment of the amino acid sequence within the other component (liposome, micelle) or the impregnation of the amino acid sequence within a polymer to produce the final composition of the present invention.

Replacements/Substitutions

The term “wherein up to 20% of amino acids of the native sequence have been replaced” refers to substitution (conservative or non conservative) with a naturally or non-naturally occurring amino acid, or with a peptidomimetic organic moiety. The term refers to an amino acid sequence which shares at least 80% of its amino acid with the native sequence as described in (i), (ii), (iv), (v), (vi), or (vii) above, but in which some of the amino acids were replaced by other naturally occurring amino acids, (both conservative and non-conservative substitutions), by non-naturally occurring amino acids (both conservative and non-conservative substitutions), or with organic moieties which serve either as true peptidomimetics (i.e. having the same steric and electrochemical properties as the replaced amino acid), or which merely serve as spacers in lieu of an amino acid, so as to keep the spatial relations between the amino acids on either side of this replaced amino acid. Guidelines for the determination of the replacements and substitutions are given in detail below. Preferably no more than about 15%, about 10% or about 5% of the amino acids are replaced.

Chemical Modification

The term “chemically modified” refers to both the chemical modification of the side chains of the amino acids as well as to chemical modifications of the peptidic backbone. It also refers to a skipping 5 peptide which has the same type of amino acid residue, but in which a functional group has been added to the side chain. For example, the side chain may be phosphorylated, glycosylated, fatty acylated, acylated, iondiated or carboxyacylated. Other examples of chemical substitutions are known in the art and given below.

The replacement may be of at least one peptidic backbone by a non-naturally occurring peptidic backbone. For example, the bond between the N— of one amino acid residue to the C— of the next has been altered to non-naturally occurring bonds by reduction (to —CH2—NH—), alkylation (methylation) on the nitrogen atom, or the bonds have been replaced by amidic bond, urea bonds, or sulfonamide bond, etheric bond (—CH2—O—), thioetheric bond (—CH2—S—), or to —CS—NH—. The side chain of the residue may be shifted to the backbone nitrogen to obtain N-alkylated-Gly (a peptidoid).

Deletions

The term “deletions” refer to an amino acid sequence which maintains at least 20% of its parental amino acid content but with at least one amino acid removed. Preferably no more than 10% of the amino acids are deleted and more preferably none of the amino acids are deleted.

The term “provided that at least 80% of the amino acids in the parent protein are maintained unaltered in the variants” preferably includes sequences in which up to 20% substitutions, up to 20% chemical modifications and up to 20% deletions are present, i.e. the same variant may have substitutions, chemical modifications and deletions so long as at least 80% of the native amino acids are identical to those of the native sequence both with regard to the nature of the amino acid residue and its position in the sequence.

Typically “essential amino acids” (essential for binding to the ligand) are maintained or replaced by conservative substitutions while non-essential amino acids may be maintained, deleted or replaced by conservative or non-conservative replacements. Essential amino acids are optionally and preferably those residues indicated at the following positions: E74, Y82, N86, D84, E114, E117 of the CD40 skipping exon 5 variant.

Addition of Groups

Fusion proteins of receptor molecules and the Fc of immunoglobulins have been shown to greater influence transmembrane signaling-related pathways than unfused receptor molecules, presumably by creating receptor dimers which are more stable than monomers (K M Mohler, et al., J. Immunol, 151, (3) 1548-1561, 1993). Addition of an Fc chain to various CD40 proteins has been shown to increase the lifetime (T1/2) of the construct, and to simplify the protein extraction procedure.

Where the composition of the invention is a linear molecule, it is possible to place various functional groups at any of its terminals. The purpose of such a functional group may be for the improvement of the CD40 ligand binding of the composition. The functional groups may also serve the purpose of improving the activity of the composition in a manner such as: improvement in stability, penetration (through cellular membranes or barriers), tissue localization, efficacy, decreased clearance, decreased toxicity, improved selectivity, improved resistance to repletion by cellular pumps, and the like. For convenience, the free N-terminus of one of the sequences contained in the compositions of the invention will be termed as the N-terminus of the composition, and the free C-terminus of the sequence will be considered as the C-terminus of the composition (these terms being used for description only and not intended to be limited in any way). Either the C-terminus or the N-terminus of the sequences, or both, can be linked to a carboxylic acid functional group or to an amine functional group, respectively.

Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the active ingredient attached thereto into a cell, for example, by reducing the hydrophilicity and increasing the lipophilicity of the active ingredient, these being an example for “a moiety for transport across cellular membranes”.

These moieties can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminus protecting groups. Carboxylic acid protecting groups include aliphatic, benzylic and aryl esters, as described above for C-terminus protecting groups. In one embodiment, the carboxylic acid group in the side chain of one or more glutamic acid or aspartic acid residue in a composition of the present invention is protected, preferably with a methyl, ethyl, benzyl or substituted benzyl ester, more preferably as a benzyl ester.

Examples of N-terminus protecting groups include acyl groups (—CO-R1) and alkoxy carbonyl or aryloxy carbonyl groups (—CO-O-R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO- and (substituted benzyl)-CO—. Examples of alkoxy carbonyl and aryloxy carbonyl groups include CH3-O-CO—, (ethyl)-O-CO—, n-propyl-O-CO—, iso-propyl-O-CO—, n-butyl-O-CO—, sec-butyl-O-CO—, t-butyl-O-CO—, phenyl-O—CO—, substituted phenyl-O-CO- and benzyl-O-CO—, (substituted benzyl)—O-CO—, adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminus protecting groups include —NH₂, —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), -O-(iso-propyl), —O-(sec-butyl), —O-(t-butyl), —O-benzyl and —O-phenyl.

Replacements by Peptidomimetic Compositions

The replacement may be also by a peptidomimetic organic moiety.

A “peptidomimetic organic moiety” can be substituted for amino acid residues in the composition of this invention both as conservative and as non-conservative substitutions. These peptidomimetic organic moieties can replace amino acid residues, amino acids or act as spacer groups within the peptides in lieu of deleted amino acids. The peptidomrnimetic organic moieties often have steric, electronic or configurational properties similar to the replaced amino acid and such peptidomimetics are used to replace amino acids in the essential positions, and are considered conservative substitutions. However such similarities are not necessarily required. The only restriction on the use of peptidomimetics is that the composition retains its physiological activity as compared to sequence regions identical to those appearing in the native protein.

Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988)); LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S,3S)-methyl-phenylalanine, (2S,3R)-methyl-phenylalanine, (2R,3S)-methyl-phenylalanine and (2R,3R)-methyl-phenylalanine (Kazmierski and Hruby, Tetrahedron Lett. (1991)).

Chemical Modifications

In the present invention the side amino acid residues appearing in the native sequence may be chemically modified, i.e. changed by addition of functional groups. The modification may be in the process of synthesis of the molecule, i.e. during elongation of the amino acid chain and amino acid, i.e. a chemically modified amino acid is added. However, chemical modification of an amino acid when it is present in the molecule or sequence (“in situ” modification) is also possible.

The amino acid of any of the sequence regions of the molecule can be modified (in the peptide conceptionally viewed as “chemically modified”) by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

Cyclization of the Molecule

The present invention also includes cyclic compounds that are cyclic molecules.

A “cyclic molecule” refers, in one instance, to a compound of the invention in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the sequence present therein, preferably the side chain of the C-terminal amino acid. For example, an amide can be formed between the nitrogen atom at the N-terminus and the carbonyl carbon in the side chain of an aspartic acid or a glutamic acid. Alternatively, the compound can be cyclized by forming a covalent bond between the carbonyl at the C-terminus of the compound and the side chain of a suitable amino acid in the sequence contained therein, preferably the side chain of the N-terminal amino acid. For example, an amide can be formed between the carbonyl carbon at the C-terminus and the amino nitrogen atom in the side chain of a lysine or an ornithine. Additionally, the compound can be cyclized by forming an ester between the carbonyl carbon at the C-terminus and the hydroxyl oxygen atom in the side chain of a serine or a threonine.

“Cyclized” also refers to forming a ring by a covalent bond between the side chains of two suitable amino acids in the sequence present in the compound, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an ornithine.

In addition, a compound can be cyclized with a linking group between the two termini, between one terminus and the side chain of an amino acid in the compound, or between the side chains to two-amino acids in the peptide or peptide derivative. Suitable linking groups are disclosed in Lobl et al., WO 92/00995 and Chiang et al., WO 94/15958, the teachings of which are incorporated into this application by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd and Acm is easily removed by iodine treatment.

The composition of the present invention can be administered parenterally. Parenteral administration can include, for example, systemic administration, such as by intramuscular, intravenous, subcutaneous, or intraperitoneal injection. Compositions that resist proteolysis can be administered orally, for example, in capsules, suspensions or tablets. The composition can also be administered by inhalation or insufflations or via a nasal spray.

The active ingredient of the invention can be administered to the individual in conjunction with an acceptable pharmaceutical carrier as part of a pharmaceutical composition for treating the diseases discussed above. Suitable pharmaceutical carriers may contain inert ingredients which do not interact with the active ingredients. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Methods for encapsulating compositions (such as in a coating of hard gelatin or cyclodextran) are known in the art (Baker et. al., Controlled Release of Biological Active Agents, John Wiley and Sons, 1986). The formation may be also resources for administration to bone, or in the form of salve, solution, ointment, etc. for topical administration.

The pharmaceutical compositions may also be administered in conjunction with other modes of therapy routinely used in the treatment of the diseases specified.

A “therapeutically effective amount” is the quantity of active ingredient which results in an improved clinical outcome as a result of the treatment compared with a typical clinical outcome in the absence of the treatment. An “improved clinical outcome” results in the individual with the disease experiencing fewer symptoms or complications of the disease, including a longer life expectancy, as a result of the treatment.

Preparation of the Active Ingredients

One having ordinary skill in the art can isolate the nucleic acid molecule that encodes the skipping 5 CD40 protein, and insert it into an expression vector using standard techniques and readily available starting materials. Use can be made of a recombinant expression vector that comprises a nucleotide sequence encoding for the amino acid sequence of SEQ ID NO: 1, or the sequence (ii) (iii), (iv) as defined above. These recombinant expression vectors are useful for transforming hosts to prepare recombinant expression systems for preparing the pharmaceutical composition of the invention.

Preparation by Recombinant Methods

As will be understood by those of skill in the art, it may be advantageous to use nucleotide sequences possessing codons other than those which naturally occur in the human genome. Codons preferred by a particular prokaryotic or eukaryotic host (Murray, E. et al. Nuc Acids Res., 17:477-508, (1989)) can be selected, for example, to increase the rate of variant product expression or to produce recombinant RNA transcripts having desirable properties, such as a longer half-life, than transcripts produced from naturally occurring sequence.

The nucleic acid sequences used to produce the amino acid sequence of the present invention can be engineered in order to alter the skipping 5 CD40 protein/peptide sequences for a variety of reasons, including but not limited to, alterations which modify the cloning, processing and/or expression of the product. For example, alterations may be introduced using techniques which are well known in the art, e.g., site-directed mutagenesis, to insert new restriction sites, to alter glycosylation patterns, to change codon preference, etc.

For producing the protein or peptide used by the present invention recombinant constructs comprising the sequence as broadly described above. The constructs may comprise a vector, such as a plasmid or viral vector, into which nucleic acid sequences coding for the protein/peptide of the invention have been inserted, in a forward or reverse orientation. In a preferred aspect of this embodiment, the constructs further comprise regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook et al., Molecular Cloning a Laboratory Manual, Second Ed. Cold Spring Harbor Press (1989) which is incorporated herein by reference.

The preparation may be achieved by host cells which are genetically engineered with the above vectors and the production of the product skipping 5 protein/peptide of the invention by recombinant techniques. Host cells are genetically engineered (i.e., transduced, transformed or transfected) with the above vectors which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the expression of the variant nucleic acid sequence. The culture conditions, such as temperature, pH and the like, are those previously used with the host cell selected for expression and will be apparent to those skilled in the art.

The host cells used in the preparation may comprise the recombinant expression vector that includes a nucleotide sequence that encodes a skipping 5 CD40 protein of SEQ ID NO: 1, and fragments and variants thereof. Host cells for use in well known recombinant expression systems for production of proteins are well known and readily available. Examples of host cells include bacteria cells such as E. coli, yeast cells such as S. cerevisiae, insect cells such as S. fugiperda, non-human mammalian tissue culture cells, chinese hamster ovary (CHO) cells and human tissue culture cells such as HeLa cells.

The nucleic acid sequences used to prepare the peptide/proteins may be included in any one of a variety of expression vectors for expressing a product. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and related sub-cloning procedures are deemed to be within the scope of those skilled in the art.

The DNA sequence in the expression vector is operatively linked to an appropriate transcription control sequence (promoter) to direct mRNA synthesis. Examples of such promoters include: LTR or SV40 promoter, the E. coli, lac or trp promoter, the phage lambda PL promoter, and other promoters known to control expression of genes in prokaryotic or eukaryotic cells or their viruses. The expression vectors also contains a ribosome binding site for translation initiation, and a transcription termiinator. The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E coli.

The vectors containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, Salmonella typhimurium; fungal cells, such as yeast; insect cells such as Drosophila and Spodoptera Sf9; animal cells such as CHO, COS, HEK 293 or Bowes melanoma; adenoviruses; plant cells, etc. The selection of an appropriate host is deemed to be within the scope of those skilled in the art from the teachings herein. The invention is not limited to any particular host cells which can be employed.

One having ordinary skill in the art can use commercial expression vectors and systems or others to produce the CD40 product of the invention using routine techniques and readily available starting materials. Thus, the desired proteins can be prepared in both prokaryotic and eukaryotic systems, resulting in a spectrum of processed forms of the protein. Expression systems containing the requisite control sequences, such as promoters and polyadenylation signals, and preferably enhancers, are readily available and known in the art for a variety of hosts. See e.g., Sambrook et al., Molecular Cloning a Laboratory Manual, Second Ed. Cold Spring Harbor Press (1989).

A wide variety of eukaryotic hosts are also now available for production of recombinant foreign proteins. As in bacteria, eukaryotic hosts may be transformed with expression systems which produce the desired protein directly, but more commonly signal sequences are provided to effect the secretion of the protein. Eukaryotic systems have the additional advantage that they are able to process introns which may occur in the genomic sequences encoding proteins of higher organisms. Eukaryotic systems also provide a variety of processing mechanisms which result in, for example, glycosylation, carboxy-terminal amidation, oxidation or derivatization of certain amino acid residues, conformational control, and so forth.

Commonly used eukaryotic systems include, but are not limited to, yeast, fungal cells, insect cells, mammalian cells, avian cells, and cells of higher plants. Suitable promoters are available which are compatible and operable for use in each of these host types as well as are termination sequences and enhancers, e.g. the baculovirus polyhedron promoter. As above, promoters can be either constitutive or inducible. For example, in mammalian systems, the mouse metallothionein promoter can be induced by the addition of heavy metal ions.

The particulars for the construction of expression systems suitable for desired hosts are known to those in the art. Briefly, for recombinant production of the protein, the DNA encoding the polypeptide is suitably ligated into the expression vector of choice. The DNA is operably linked to all regulatory elements which are necessary for expression of the DNA in the selected host. One having ordinary skill in the art can, using well known techniques, prepare expression vectors for recombinant production of the polypeptide.

The expression vector including the DNA that encodes the CD40 skipping 6 protein, fragment or homolog, preferably including DNA coding for the Fc fragment attached to the CD40 skipping 5 protein, is used to transform the compatible host which is then cultured and maintained under conditions wherein expression of the foreign DNA takes place. The protein of the present invention thus produced is recovered from the culture, either by lysing the cells or from the culture medium as appropriate and known to those in the art. One having ordinary skill in the art can, using well known techniques, isolate the CD40 product that is produced using such expression systems. The methods of purifying the CD40 skipping 5 protein from natural sources using antibodies which specifically bind to the skipping 5 protein, may be equally applied for purifying the product produced by recombinant DNA methodology.

Examples of genetic constructs include the skipping 5 CD40 protein coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes the skipping 5 protein from readily available starting materials.

In bacterial systems, a number of expression vectors may be selected depending upon the use intended for the CD40 product. For example, when large quantities of CD40 skipping 5 splice variant product are needed, such as for the induction of antibodies, vectors which direct high level expression of fusion proteins that are readily purified may be desirable. Such vectors include, but are not limited to, multifunctional E. coli cloning and expression vectors such as Bluescript(R) (Stratagene), in which the CD40 splice variant polypeptide coding sequences may be ligated into the vector in-frame with sequences for the amino-terminal Met and the subsequent 7 residues of beta-galactosidase so that a hybrid protein is produced; pIN vectors (Van Heeke & Schuster J. Biol. Chem. 264:5503-5509, (1989)); pET vectors (Novagen, Madison Wis.); and the like. In some embodiments, for example, one having ordinary skill in the art can, using well known techniques, insert such DNA molecules into a commercially available expression vector for use in well known expression systems. For example, the commercially available plasmid pSE420 (Invitrogen, San Diego, Calif.) may be used for production of collagen in E. coli.

In the yeast Saccharomyces cerevisiae a number of vectors containing constitutive or inducible promoters such as alpha factor, alcohol oxidase and PGH may be used. For reviews, see Ausubel et al. (Supra) and Grant et al., (Methods in Enzymology 153:516-544, (1987)). The commercially available plasmid pYES2 (Invitrogen, San Diego, Calif.) may, for example, be used for production in S. cerevisiae strains of yeast.

In cases where plant expression vectors are used, the expression of a sequence encoding variant products may be driven by any of a number of promoters. For example, viral promoters such as the 35S and 19S promoters of CaMV (Brisson et al., Nature 310:511-514. (1984)) may be used alone or in combination with the omega leader sequence from TMV (Takamatsu et al, EMBO J., 6:307-311, (1987)). Alternatively, plant promoters such as the small subunit of RUBISCO (Coruzzi et al., EMBO J. 3:1671-1680, (1984); Broglie et al., Science 224:838-843, (1984)); or heat shock promoters (Winter J and Sinibaldi R. M., Results Probl. Cell Differ., 17:85-105, (1991)) may be used. These constructs can be introduced into plant cells by direct DNA transformation or pathogen-mediated transfection. For reviews of such techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

CD40 splice variant products may also be expressed in an insect system. In one such system, Autographa californica nuclear polyhedrosis virus (AcNPV) is used as a vector to express foreign genes in Spodoptera frugiperda cells or in Trichoplusia larvae. The CD40 skipping 5 coding sequence may be cloned into a nonessential region of the virus, such as the polyhedrin gene, and placed under control of the polyhedrin promoter. Successful insertion of the skipping 5 coding sequence will render the polyhedrin gene inactive and produce recombinant virus lacking coat protein coat. The recombinant viruses are then used to infect S. frugiperda cells or Trichoplusia larvae in which variant protein is expressed (Smith et al., J. Virol. 46:584, (1983); Engelhard, E. K. et al., Proc. Nat. Acad. Sci. 91:3224-7, (1994)). The commercially available MAXBACJ complete baculovirus expression system (Invitrogen, San Diego, Calif.) may, for example, be used for production in insect cells.

In mammalian host cells, a number of viral-based expression systems may be utilized. In cases where an adenovirus is used as an expression vector, the skipping 5 CD40 coding sequences may be ligated into an adenovirus transcription/translation complex consisting of the late promoter and tripartite leader sequence. Insertion in a nonessential E1 or E3 region of the viral genome will result in a viable virus capable of expressing variant protein in infected host cells (Logan and Shenk, Proc. Natl. Acad. Sci. 81:3655-59, (1984). In addition, transcription enhancers, such as the Rous sarcoma virus (RSV) enhancer, may be used to increase expression in mammalian host cells. The commercially available plasmid pcDNA I (Invitrogen, San Diego, Calif.) may, for example, be used for production in mammalian cells such as Chinese Hamster Ovary cells.

Specific initiation signals may also be required for efficient translation of product coding sequences. These signals include the ATG initiation codon and adjacent sequences. In cases where the CD40 sequence, its initiation codon and upstream sequences are all inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al., (1994) Results Probl. Cell Differ., 20: 125-62, (1994); Bitner et al, Methods in Enzymol 153:516-544, (1987)).

The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., and Battey, I. (1986) Basic Methods in Molecular Biology). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre-pro” form of the protein may also be important for correct insertion, folding and/or function. Different host cells such as CHO, HeLa, MDCK, 293, W138, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express skipping 5 may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant colonies of stably transformed cells can be proliferated using tissue culture techniques appropriate to the cell type.

Any number of selection systems may be used to recover transformed cell lines. These include, but are not limited to, the herpes simplex virus thymidine kinase (Wigler M., et al., Cell 11:223-32, (1977)) and adenine phosphoribosyltransferase (Lowy I., et al., Cell 22:817-23, (1980)) genes which can be employed in tk- or aprt-cells, respectively. Also, antimetabolite, antibiotic or herbicide resistance can be used as the basis for selection; for example, dhfr which confers resistance to methotrexate (Wigler M., et al., Proc. Natl. Acad. Sci. 77:3567-70, (1980)); npt, which confers resistance to the aminoglycosides neomycin and G-418 (Colbere-Garapin, F. et al., J. Mol. Biol, 150: 1-14, (1981)) and als or pat, which confer resistance to chlorsulftiron and phosphinotricin acetyltransferase, respectively (Murry, Supra). Additional selectable genes have been described, for example, trpB, which allows cells to utilize indole in place of tryptophan, or hisD, which allows cells to utilize histinol in place of histidine (Hartman S. C. and R. C. Mulligan, Proc. Natl. Acad. Sci 85:8047-51, (1988)). The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system (Rhodes, C. A. et al., Methods Mol. Biol., 55:121-131, (1995)).

Host cells transformed with nucleotide sequences encoding the skipping 5 or its fragments may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors can be designed with signal sequences which direct secretion of the skipping 5 CD40 product through a prokaryotic or eukaryotic cell membrane.

The present invention encompasses use of a transgenic non-human mammal that comprises the recombinant expression vector that comprises a nucleic acid sequence encoding for the CD40 splice variant of amino acid sequence SEQ ID NO:1; and fragments and homologues thereof. Transgenic non-human mammals useful to produce recombinant proteins are well known as are the expression vectors necessary and the techniques for generating transgenic animals. Typically, the transgenic animal comprises a recombinant expression vector in which the nucleotide sequence that encodes the skipping 5 CD40 protein of the invention is operably linked to a mammary cell specific promoter whereby the coding sequence is only expressed in mammary cells and the recombinant protein expressed is recovered from the animal's milk. One having ordinary skill in the art using standard techniques, such as those taught in U.S. Pat. No. 4,873,191 issued Oct. 10, 1989 to Wagner and U.S. Pat. No. 4,736,866 issued Apr. 12, 1988 to Leder, both of which are incorporated herein by reference, can produce transgenic animals which produce the CD40 product of the present invention. Preferred animals are rodents, particularly, rats and mice, or goats.

In some embodiments, the skipping 5 protein may be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle, Wash.).

The inclusion of a protease-cleavable polypeptide linker sequence between the purification domain and the skipping 5 protein, is useful to facilitate purification. One such expression vector provides for expression of a fusion protein compromising a variant polypeptide fused to a polyhistidine region separated by an enterokinase cleavage site. The histidine residues facilitate purification on IMIAC (immobilized metal ion affinity chromatography, as described in Porath, et al., Protein Expression and Purification, 3:263-281, (1992)) while the enterokinase cleavage site provides a means for isolating variant polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, then disrupted by physical or chemical means, and the resulting crude extract retained for further purification. Microbial cells employed in expression of proteins can by disrupted by any convenient method, including freeze-thaw cycling, sonication, mechanical disruption, or use of cell lysing agents, or other methods, which are well know to those skilled in the art.

Purification of Recombinant Produced Peptide/Proteins

The CD40 skipping 5 product can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps. In some embodiments, antibodies may be used to isolate the skipping 5 proteins.

Production by Synthesizers

In addition to producing these proteins by recombinant techniques, automated peptide synthesizers may also be employed to produce the CD40 skipping 5 proteins, fragments or homologues of the invention. Such techniques are well known to those having ordinary skill in the art and are useful if derivatives which have substitutions not provided for in DNA-encoded protein production. CD40 skipping 5, fragments and portions of the products may be produced by direct peptide synthesis using solid-phase techniques (cf. Stewart et al., (1969) Solid-Phase Peptide Synthesis, WH Freeman Co, San Francisco; Merrifield J., J. Am Chem. Soc., 85:2149-2154, (1963)). In vitro peptide synthesis may be performed using manual techniques or automation. Automated synthesis may be achieved, for example, using Applied Biosystems 431A Peptide Synthesizer (Perkin Elmer, Foster City, Calif.) in accordance with the instructions provided by the manufacturer. Fragments of the skipping 5 CD40 protein may be chemically synthesized separately and combined using chemical methods to produce the full length molecule.

Pharmaceutical Compositions and Methods of Administration.

Each of the upregulating or downregulating agents described hereinabove or the expression vector encoding CD40 can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that treatment of CD40 related disease according to the present invention may be combined with other treatment methods known in the art (i.e., combination therapy).

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization —A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Experimental data show that the novel sequences of the invention have an unexpected biochemical effect. In particular, the data presented below show that while a known truncation product derived from known CD40, lacking the unique sequence spanning the amino acids 136-160 of the sequence depicted in SEQ ID NO: 1, caused a decreased level of cytokine RANTES secretion, the administration of the CD40 skipping exon 5 variant of the present invention resulted in an increased level of RANTES secretion. This finding is evidence that the skipping 5 variant of human CD40 of the invention is novel and non-obvious since it shows unexpected effects, which are different from those of state of the art truncated CD40 partial proteins.

### Example 1

Production of Skipping 5 Polyclonal Antibody

Rabbit was immunized with KLH conjugated 95% purified RPKTWLCNRQAQTRLMLS polypeptide (SEQ ID NO:6), located at the unique tail of CD40 skipping 5 splice variant product, VRPKTWLCNRQAQTRLMLSVVPRIG (SEQ ID NO: 5).

The anti-CD40-skipping 5 antibodies were then purified from rabbit serum by ammonium sulfate precipitation. Briefly, a saturated solution of ammonium sulfate was prepared by adding 380 gr to 500 ml water and boiling the solution. The serum was thawed and centrifuged at 10,000 rpm, 4° C. for 5 min. One vol. PBS was added to each vol. serum, and stirred at 4° C.

One volume of saturated ammonium sulfate was then added under stirring for at least 2 hours on ice. The solution was centrifuged 15 min. at 10,000 rpm at 4° C. to precipitate IgG. The pellet was resuspended in 5 ml PBS and dialyzed overnight at 4° C. against PBS+0.05% azide. The precipitated serum was filtered with a 0.45 cm filter.

Affinity purification was then performed with the peptide against which the respective antibodies were raised as described above, in an immunoaffinity column, linked to sulfolink beads (Pierce # 20401). The column was prepared according to manufacturer's instructions. The serum to be purified was mixed with sulfolink beads and incubated under gentle shaking (1 h at room temperature (RT) and 2 h at 40° C.), after which the beads were packed into a column.

The column was washed with TRIS 100 mM, followed by binding buffer containing 0.5M NaCl. The IgG was eluted by applying elution buffer: 0.1M Glycine pH3 (fraction size: 0.5 ml), followed by phosphate buffer 100mM pH11 to elute another fraction of IgG. In order to neutralize acidic or basic pH, 1/10 volume TRIS 1M pH8 was added to collecting tubes before addition of elution buffer to the column. The antibodies were dialyzed overnight against a buffer of PBS and 0.025% azide, and then frozen for storage.

Measurement of Peptide Concentration:

2 mg of lyophilized peptide were diluted into 1 ml DDW (double distilled water) and the absorbance at A₂₈₀ was measured. 2 ml of slurry sulfolink gel at room temperature were added to a column and the buffer was drained.

The column was equilibrated with 6 volumes of coupling buffer, containing 50 mM Tris, 5 mM EDTA pH 8.5. 5 ml coupling buffer containing 1 mM β-mercaptoehtanol (3.5 μl β-mercaptoethanol 98% to 50 ml TRIS buffer) were added to the peptide, following the addition of the beads and mix at RT for 15 min. Additional incubation for another 30 min at RT without mixing followed.

Next, the buffer was drained, kept and measured for absorbance to check for unbound peptide. The absorbance was measured as follows: A₂₈₀=0.2.

Blocking was performed by the addition of p-mercaptoethanol (100 mM) to the coupling buffer (35 μl β-mercaptoethanol to 5 ml buffer) and mixing at RT for 15 min, following incubation at RT for 30 min without mixing. Following the washing with the coupling buffer without β-mercaptoehtanol and then with NaCl 1M, the column was kept in PBS or TRIS containing 0.05% NaN₃.

Purification Using FPLC (Fast Protein Liquid Chromatography):

Before the first use of the columns, all of the buffers used for purification were passed through the column, in order to remove any remaining impurities in the beads.

Into each collecting tube, 1/10 collecting volume of TRIS 1M pH 8 and ice were added. The column was washed and kept in the binding buffer (PBS) until the antibody-containing sample was loaded. The precipitated serum was filtered, mixed with sulfolink beads and incubated under gentle shaking (1 h at R.T. and 2 h at 4° C.). The beads were packed into the column and washed with TRIS 100 mM, and were then washed with binding buffer containing 0.5M NaCl. The eluted fractions were kept for further analyses (to be certain that they do not contain the Ab). Purified IgG was eluted by applying elution buffer: 0.1M Glycine pH3 (fractions size: 0.5 ml). Each collected fraction was mixed immediately with the 1M TRIS buffer (1:10) in the collecting tube. The column was washed with binding buffer. Phosphate buffer 100 mM pH 11 was applied to elute another fraction of IgG. The absorption of the relevant fractions was measured at OD A₂₈₀ (GeneQuant: use the DNA channel) (calculation of concentration: A₂₈₀=n; C=n/1.35 mg/ml) and dialyzed o.n. (overnight) against PBS+0.025% azide.

The column was regenerated and stored as follows: washed with PBS 50 mM+0.025% azide and stored at 4° C.

The antibodies were aliquoted and stored in one of three different solutions:4° C. with 1% BSA and sodium azide (0.025%); −20° C. with 1% BSA, 50% glycerol and sodium azide (0.025%); or −70° C. with 1% BSA and sodium azide (0.025%).

### Example 2

Cloning of CD40-Skipping 5 Variant and the known CD40

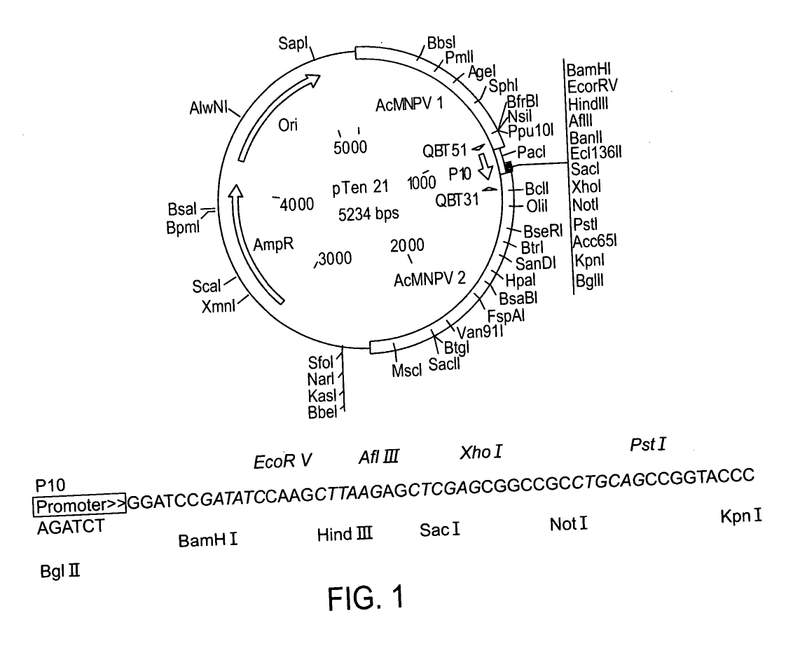

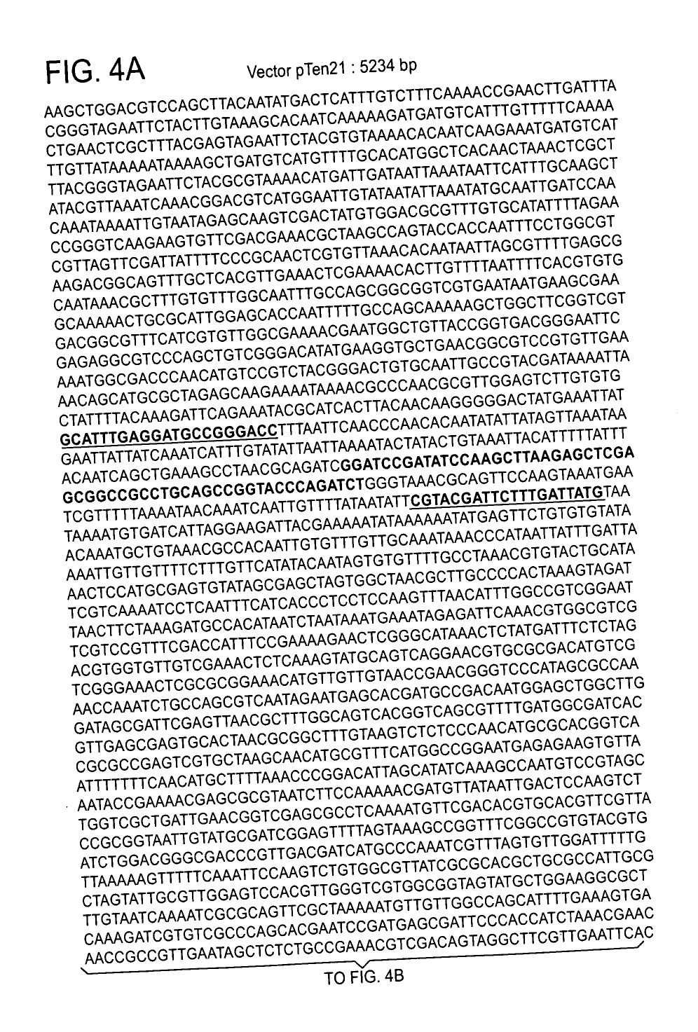

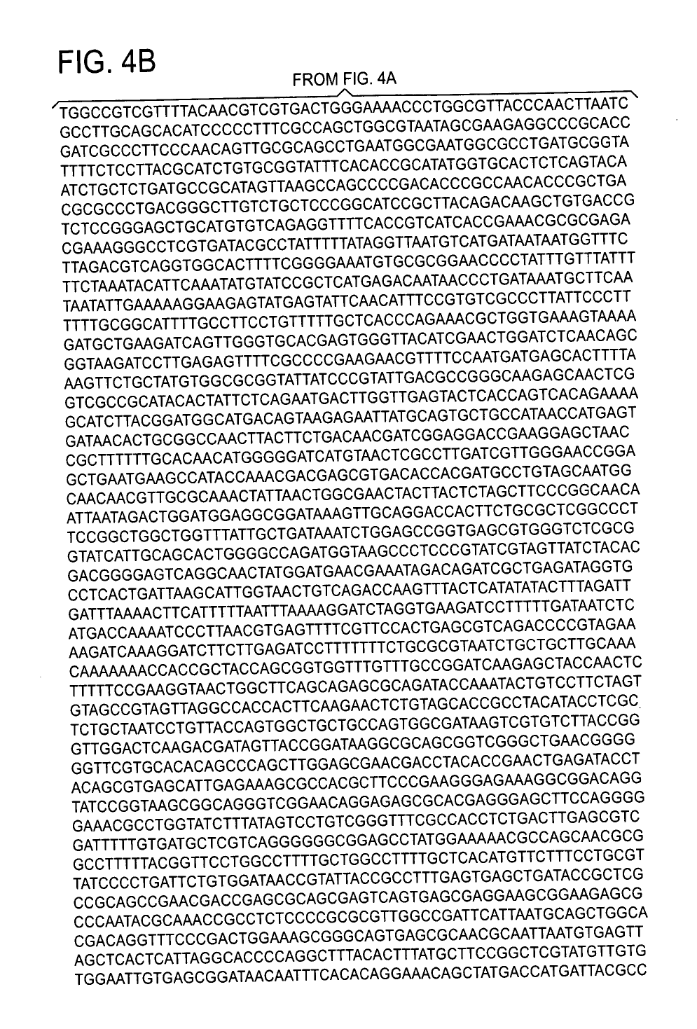

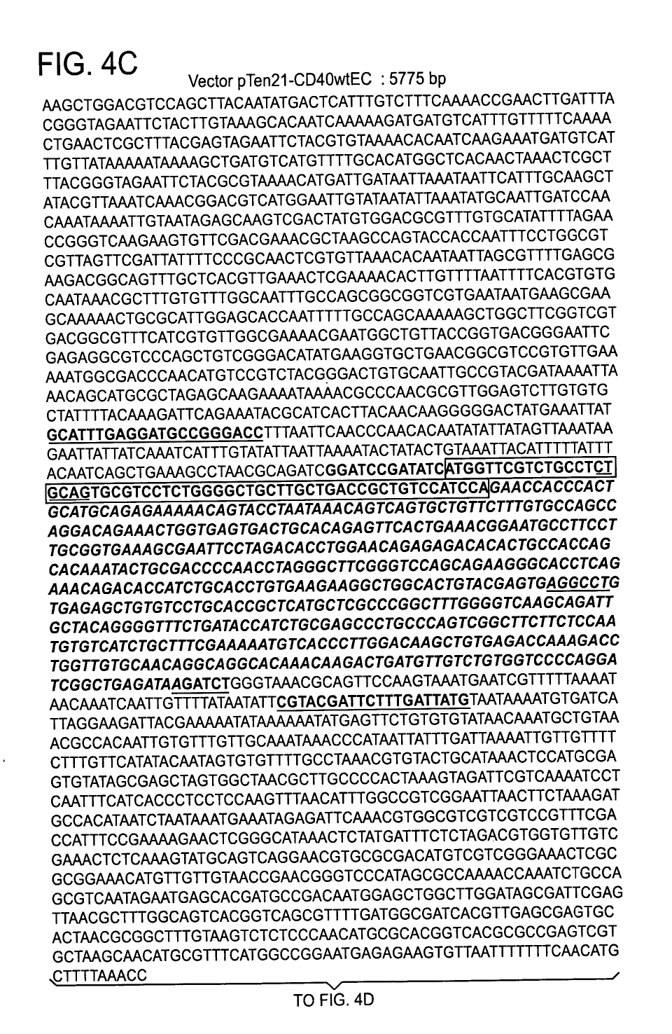

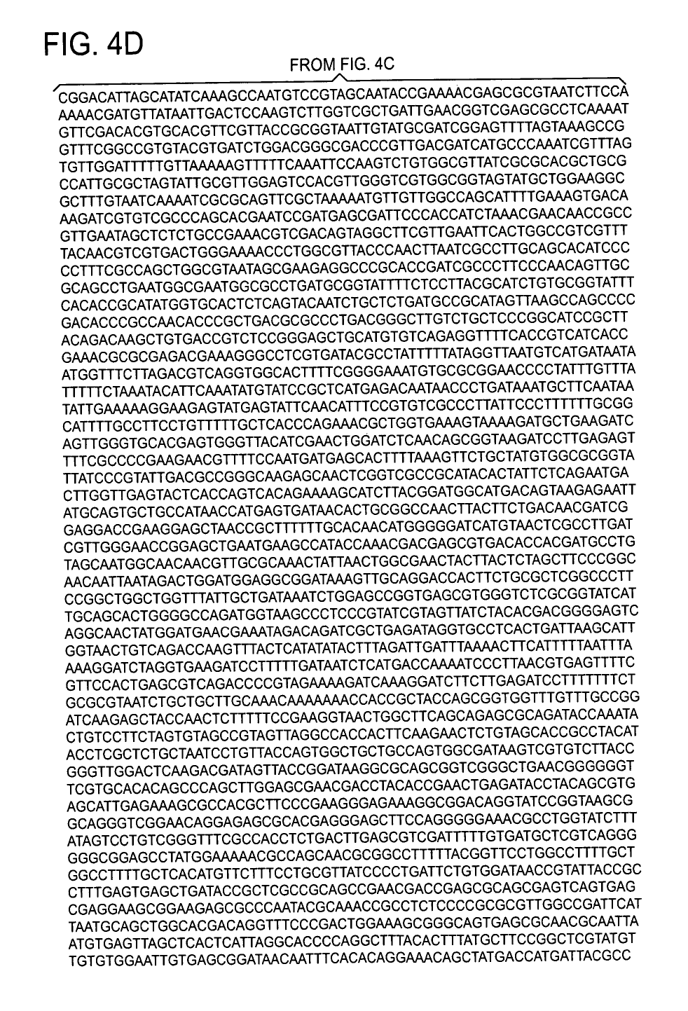

For all of the constructed transfer vectors, the backbone was the pTen21 plasmid whose full-length sequence is given in SEQ ID NO: 10 and in FIG. 4a. The plasmid map and its multiple cloning site sequence are given in FIG. 1.

1—Construction of pTen21-CD40 wtEC Vector:

The known CD40 extracellular domain sequence was amplified by PCR from the provided plasmid using the following primers (the transmembrane domain of the known CD40 protein was excluded, therefore this fragment of the known CD40 protein, upon translation, will be secreted; it should be noted that this process results in a protein that is a simple truncation product of known CD40). The PCR amplification of the known CD40 extracellular domain was carried out with the following primers: SEQ ID NO:7, called 40 wt5′

5′-ACTAgATATCATggTTCgTCTgCCTCTgCAgT-3′ SEQ ID NO:8, called 40 wt3′

5′-AAgCAgATCTTATCTCAgCCgATCCTgggg-3′

The PCR reaction was carried out using the ISIS DNA polymerase (Qbiogene Cat# EPSIS100).

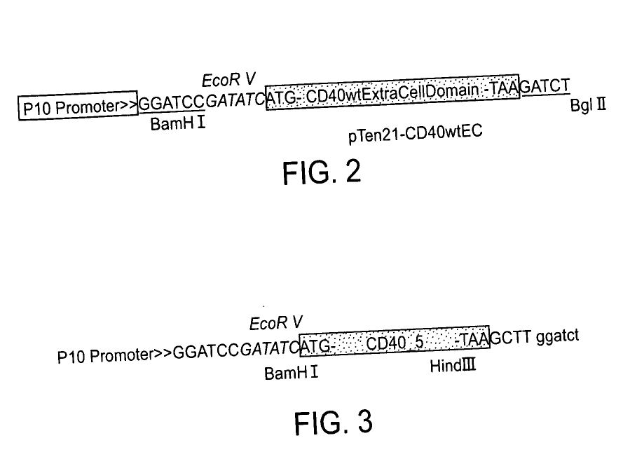

The amplified fragment of about 600 bp was digested with EcoRV and BglII and ligated to pTen21 vector previously cut with the same enzymes to obtain the vector as described in FIG. 2.

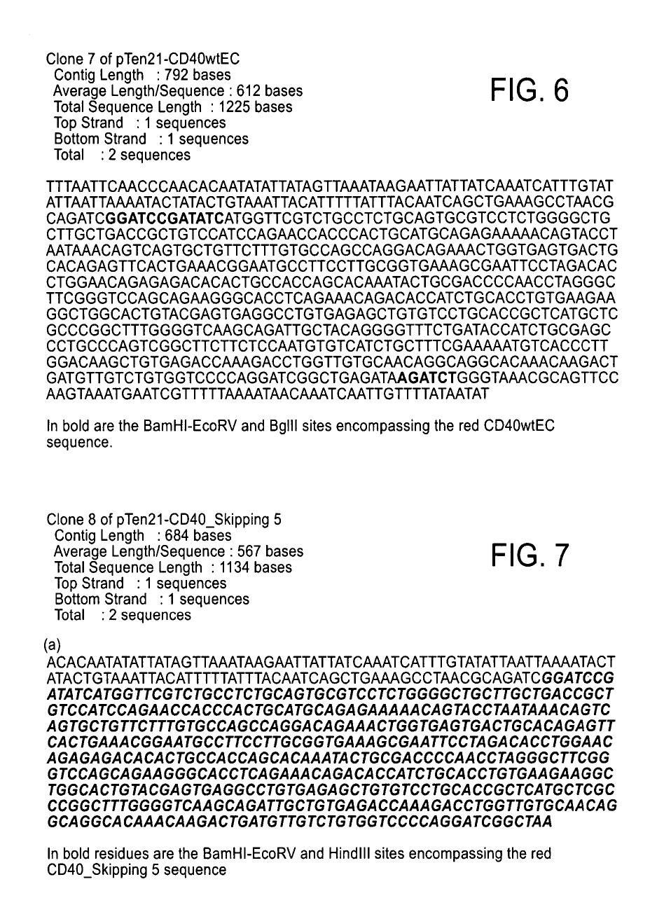

Among the sequenced recombinant constructs, clone number 7 was selected. The bi-directional sequencing was performed using the upstream primer OQBT51 (5′-GCATTTGAGGATGCCGGGACC-3′; (SEQ ID NO:11)) and the downstream primer OQBT31 (5′-CATAATCAAAGAATCGTACG-3′; (SEQ ID NO:12)). The positions of these 2 primers are indicated (bold & underlined) on the vector sequences in FIG. 4. The sequence determined for Clone 7 is shown in SEQ ID NO:9 and in FIG. 6, featuring the BamHI-EcoRV and BglII sites marked in bold, encompassing the CD40 wtEC sequence.

2—Construction of pTen21-CD40-Skipping 5 Vector:

Using the provided pET28a-CD40_{—}5 plasmid, a sequence extending from the internal StuI site to the Stop codon of CD40_Skipping 5 variant has been amplified by PCR. For this purpose, the following primers were used:

| | | |
|---|---|---|
| 5′- CACCATCTgCACCTg | (SEQ ID NO:23) | (called Stu40) |
| TgAAg -3′ | | |
| 5′- gTAAggATCCAAgCT | (SEQ ID NO:17) | (called 4053′) |
| TAgCCgATCCTggggACCA | | |
| C -3′ | | |

The amplified fragment of about 200 bp was digested with StuI and BamHI and ligated to the previously constructed pTen21-CD40 wtEC clone 7 vector cut with StuI and BglII to obtain the vector as described in FIG. 3.

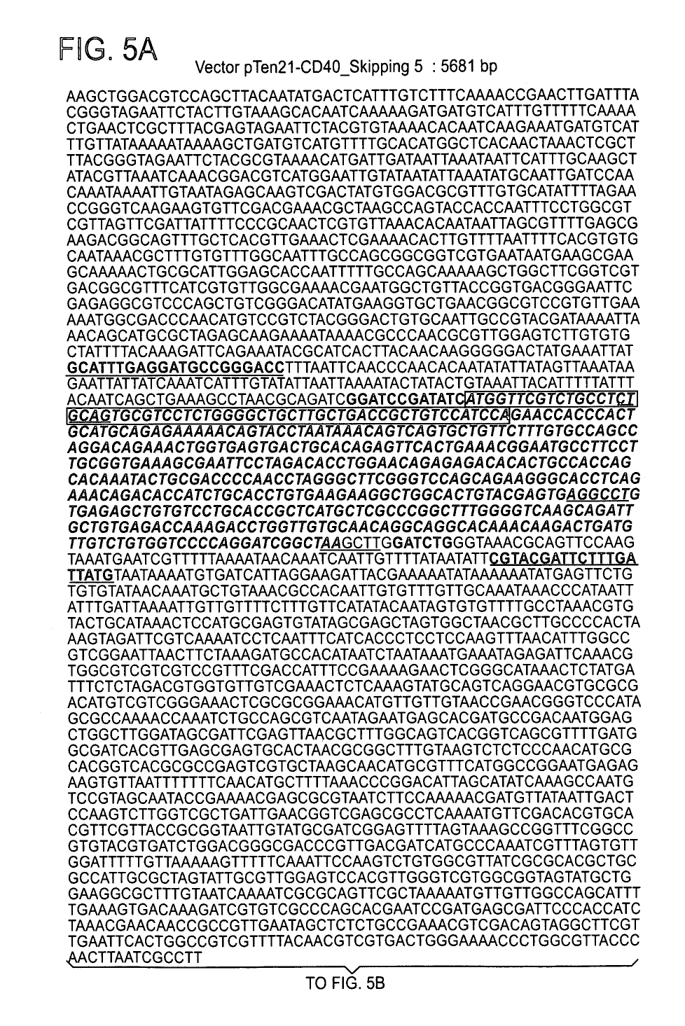

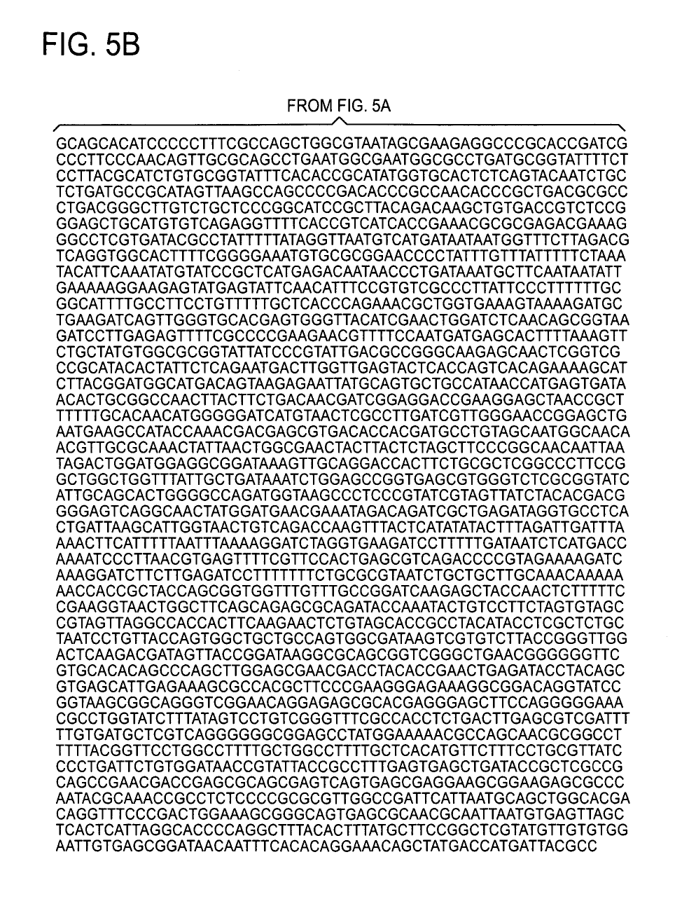

A series of recombinant plasmids were selected and sequenced with OQBT51 (SEQ ID NO:11) and OQBT31 (SEQ ID NO:12) primers. Clones 7, 8, 10 and 12 were found to be correct. For example, the sequence obtained for clone 8 is described in FIG. 7, and in SEQ ID NO:14. FIG. 5 shows the Vector pTen21-CD40_Skipping 5 full length sequence. The primers are marked in bold and underlined, in bold italic is the CD40_Skipping 5 coding sequence. Also shown are BamHI-EcoRV and BglII sites. The signal peptide is presented in the rectangle.

3—Construction of pTen21-CD40 wtEC-Fc Vector:

Fused constructs were then created, in which the Fc chain of Immunoglobulin IgG1 was fused downstream from the CD40 protein (either downstream of skipping 5 sequence, or downstream of the WT CD40). Fusion proteins of receptor molecules and the Fc of immunoglobulins have been shown to have greater influence on transmembrane signaling-related pathways than unfused receptor molecules, presumably by creating receptor dimers which are more stable than monomers (K M Mohler, et al., J. Immunol, 151, (3) 1548-1561, 1993). Addition of an Fc chain to various CD40 proteins has been shown to increase the lifetime (T_{1/2}) of the construct, and to simplify the protein extraction procedure.

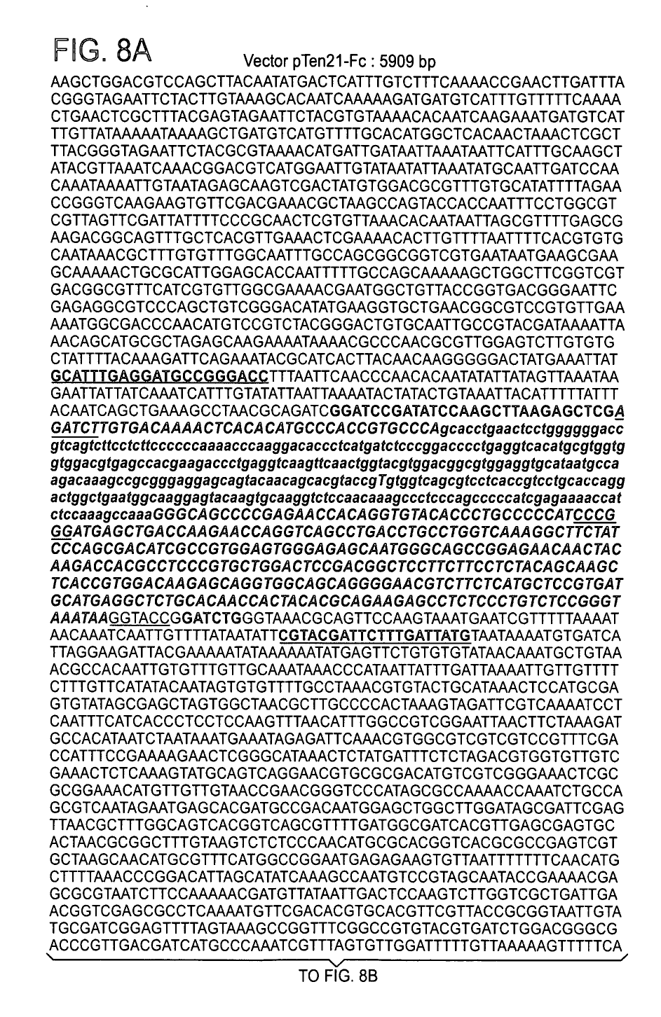

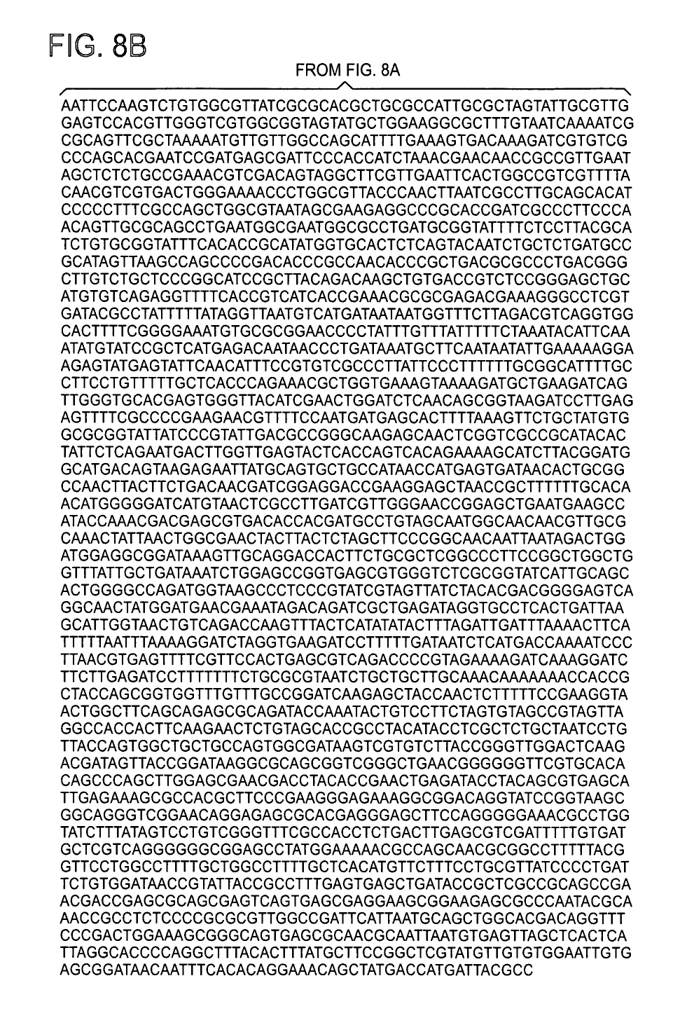

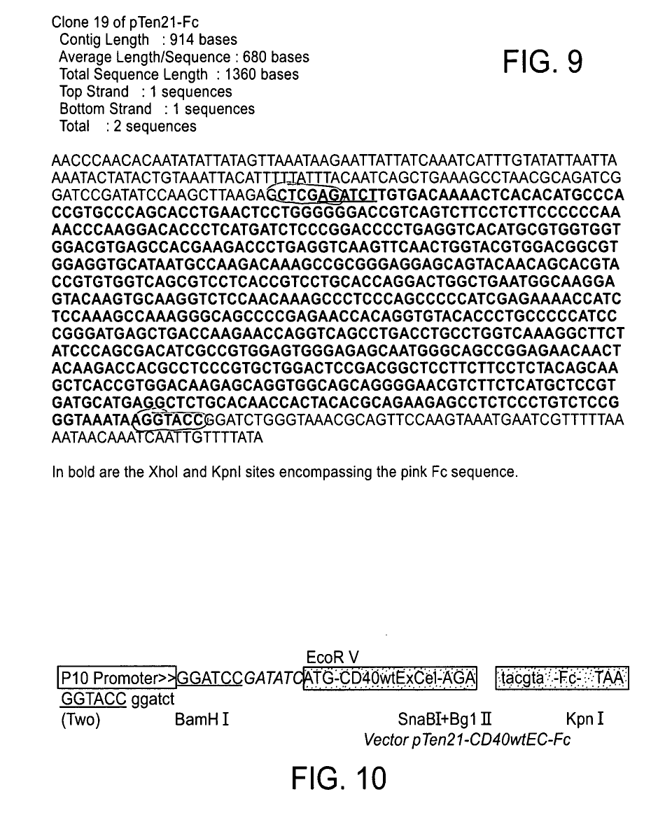

To create the Fc-fused CD40 vectors, the pTen21-Fc vector was used. The sequence of the full length pTen21-Fc vector is shown in SEQ ID NO: 13 and in FIG. 8, the OQBT primers are marked in bold and underlined and the Fc sequence is colored in bold italics. The polylinker is also shown. The Fc sequence within the pTen21-Fc vector is shown in SEQ ID NO: 15 and in FIG. 9, featuring the XhoI and KpnI sites marked in bold, encompassing the Fc sequence, which is shown in bold italics.

To construct the pTen21-CD40 wtEC-Fc fusion transfer vector, the known CD40 extracellular domain sequence has been amplified by PCR using the following primers:

5′-ACTAgATATCATggTTCgTCTgCCTCTgCAgT-3′ (SEQ ID NO:7) (called 40 wt5′)

5′-CACAAgATCTgggCTCTACgTATCTCAgCCgATCCTgggg-3′ (SEQ ID NO:16) (Called 40Fc3′)

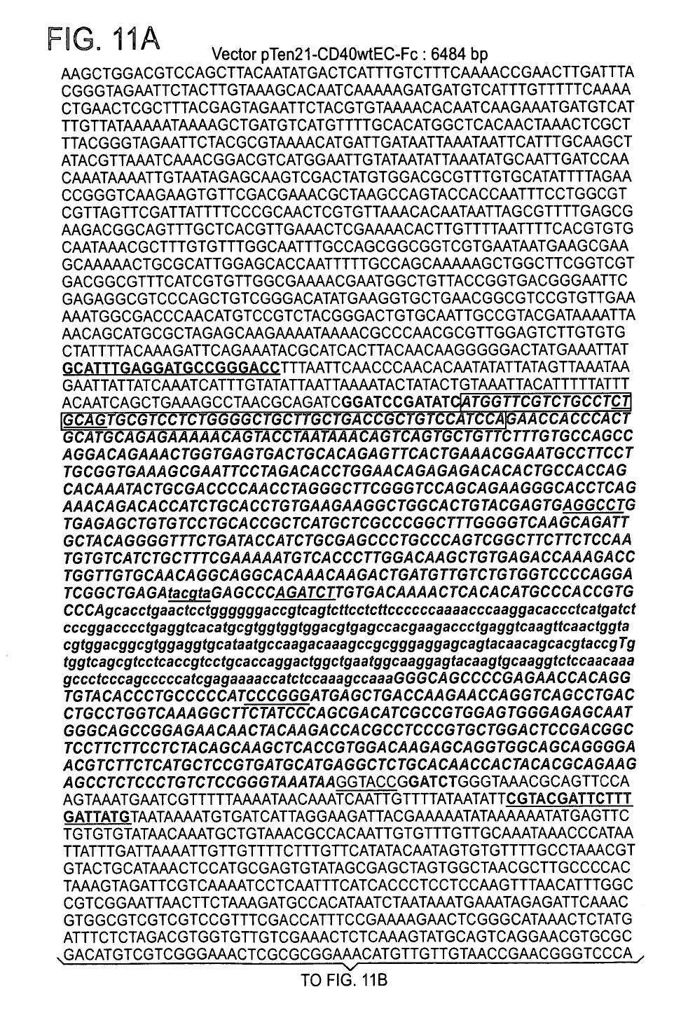

The amplified fragment of about 610 bp was digested with EcoRV and BglII and ligated to a pTen21-Fc clone 19 vector that was also cut with the same enzymes to obtain the vector as described in FIG. 10.

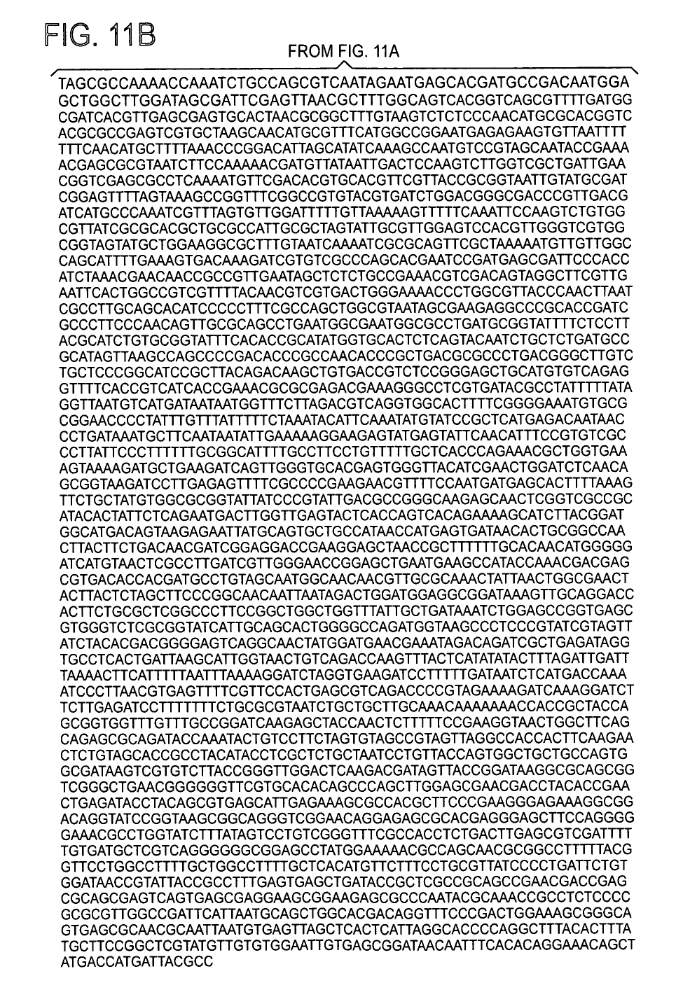

A series of recombinant plasmids were selected and sequenced using the OQBT51 (SEQ ID NO:11), OQBT31 (SEQ ID NO:12) and the internal Stu40 (SEQ ID NO:23) primer. Among them, clones 30 and 37 presented 100% homology when aligned with the corresponding portion of the expected sequence of pTen21-CD40wtEC-Fc vector shown in SEQ ID NO:18 and in FIG. 11, where primers are in bold and underlined, the CD40 wtEC-Fc fusion sequences are shown in bold italics, and are separated by the tacgta sequence.

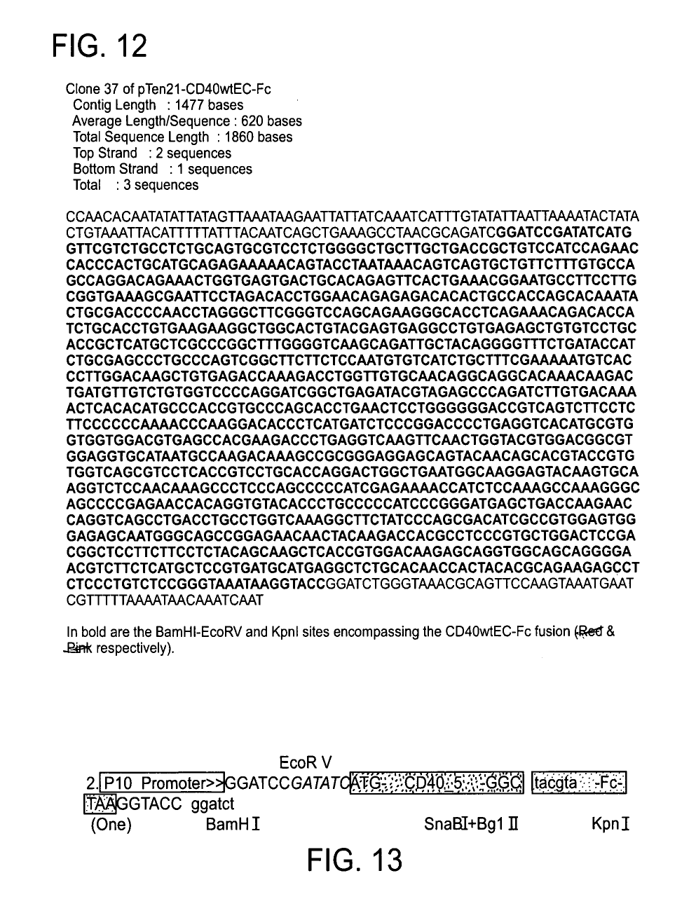

The sequence obtained for clone 37 presented in SEQ ID NO:19 and in FIG. 12, featuring the BamHI-EcoRV and KpnI sites marked in bold. The CD40wtEC-Fc fusion sequences are shown in bold italics, and are separated by the tacgta sequence.

4—Construction of pTen21-CD40 Skipping 5-Fc Vector:

Using the pET28a-CD40_{—}5 plasmid, a sequence extending from the internal StuI site to the end of CD40_Skipping 5 variant was amplified by PCR. The following primers were used:

(SEQ ID NO:23) 5′-CACCATCTgCACCTgTgAAg-3′

(Called Stu40); and (SEQ ID NO:20)5′-CACAAgATCTgggCTCTACgTAgCCgATCCTggggACCA-3′ (Called 5Fc3′).

The 200 bp amplified fragment was digested with StuI and BglII and inserted into the previously constructed pTen21-CD40wtEC-Fc clone 37 vector cut with the same enzymes to obtain the vector presented in FIG. 13.

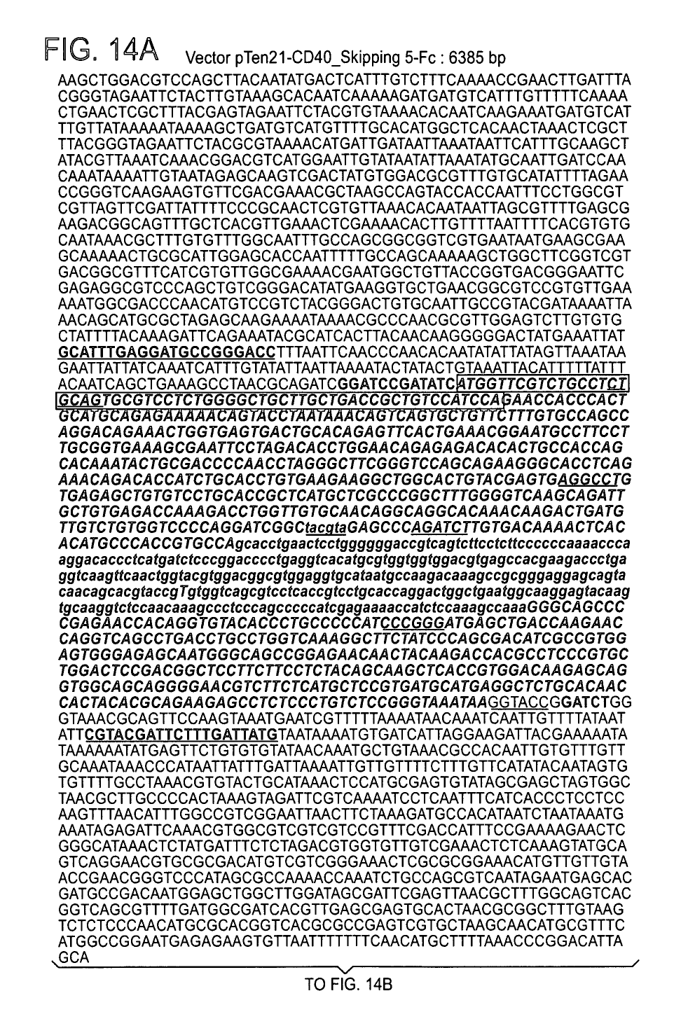

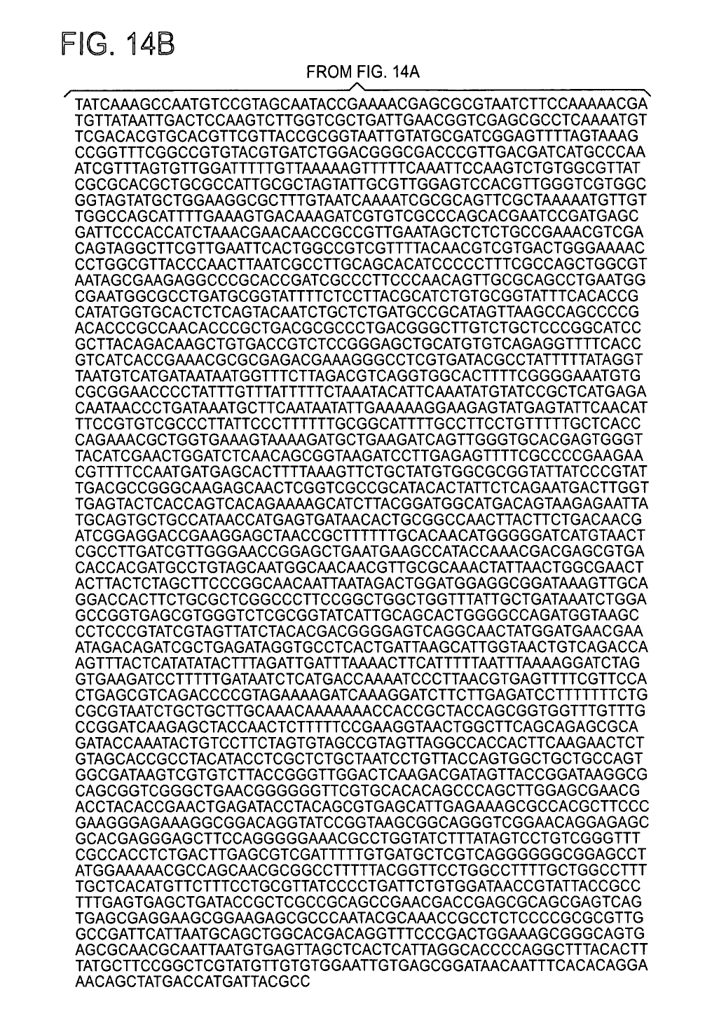

Selected plasmids were sequenced with OQBT51 (SEQ ID NO: 11), OQBT31 (SEQ ID NO:12) and the internal Stu40 (SEQ ID NO:23) primer. Clones 5 and 9 presented 100% homology with the corresponding portion of the expected sequence of pTen21-CD40_Skipping 5-Fc vector given in SEQ ID NO:21 and in FIG. 14, where primers are in bold and underlined, the CD40-skipping 5-Fc fusion are shown in bold italics, and are separated by the tacgta sequence. Also shown are the polylinker and the signal peptide-encoding sequence, which is shown with a rectangle.

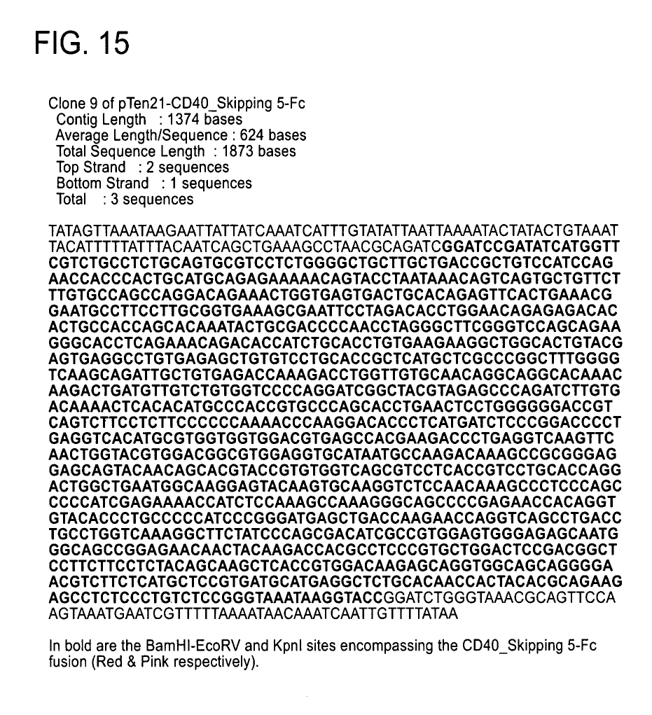

The sequence obtained for clone 9 is presented in SEQ ID NO: 22 and in FIG. 15, where the internal StuI, BamHI and BglII sites are underlined. The BamHI and EcoRV sites upstream of the ATG are in Bold. CD40-skipping 5 sequence-Fc fusion sequences are shown in bold italics, and are separated by the tacgta sequence.

### Example 3

Protein Production and Processing in Baculovirus

Protein Production and Processing from 1 L volume of Baculovirus

Baculovirus cells were transfected with the above constructs (BacTen-CD40wtEC-Fc, BacTen-CD40_Skip5-Fc, BacTen-CD40wtEC and BacTen-CD40_Skipping 5, corresponding to pTen21-CD40wtEC, pTen21-CD40_Skipping 5, pTen21-CD40wtEC-Fc and pTen21-CD40_Skipping 5-Fc, respectively), and similar constructs containing the CD40-skipping 6 variant (BacTen-CD40_Skipping 6-Fc and BacTen-CD40 Skipping 6), described in greater detail in PCT application number PCT/US2005/006531, by the inventors, herein fully incorporated by reference, and cultured to produce the expressed protein. The baculoviral culture conditions are described in table 4 below. The initial cell density, the MOI used and the harvesting time in each experiment are indicated in Table 4. Where indicated in Table 4, the anti-protease treatment was applied in cell culture medium at the following final concentrations: Pepstatin 10 μM, Leupeptin 2 μM, Pefabloc 1 mM. The final viability is indicated in Table 4 for each construct.

TABLE 4
Protein Productions and Processing from 1 L volume of Baculovirus
Culture Conditions

| | Initial cell | | Harvesting | Anti- | Final |
|---|---|---|---|---|---|
| Construct | density | MOI | Time | Proteases* | Viability |
| BacTen- | ˜800.000/ml | 2 | 72 hpi | − | 85% |
| CD40wtEC-Fc | | | | | |
| BacTen- | ˜800.000/ml | 2 | 72 hpi | + | 74% |
| CD40_Skipping 5-Fc | | | | | |
| BacTen- | ˜800.000/ml | 2 | 72 hpi | + | 80% |
| CD40_Skipping 6-Fc | | | | | |
| BacTen-CD40wtEC | ˜800.000/ml | 2 | 96 hpi | + | 92% |
| BacTen- | ˜800.000/ml | 2 | 72 hpi | + | ND |
| CD40_Skipping 5 | | | | | |
| BacTen- | ˜800.000/ml | 2 | 96 hpi | + | 75% |
| CD40_Skipping 6 | | | | | |

*Anti-proteases in cell culture medium at the following final concentrations: Pepstatin 10 μM, Leupeptin 2 μM, Pefabloc 1 mM.

Each collected 1 litre of supernatant was concentrated at 4° C. by tangential flow ultrafiltration in a 10 kDa cut-off Vivaflow 200 device (Vivascience cat# VF20PO). They were then dialysed in the same device against 50 mM Tris-HCl buffer pH 8.0. The final volume collected was around 25 ml. Solutions were then either frozen (non-fused constructs) or passed through a protein A column for purification, as described below in Example 4.

### Example 4

Purification of C-Terminus Fc-Tagged CGEN40 Variant Proteins:

The Fc-tagged proteins expressed using the baculovirus system were purified through a protein A column. The following reagents, resins and buffers were used:

Reagents:

Albumin Standard {PIERCE, Cat #23209}

Bradford reagent {BIO-RAD, Cat # 500006}

Citric acid monohydrate {MERCK, Cat # K91107244}

Dulbecoo's Phosphate Buffered Saline, concentrate X10 {Biological Industries, Cat # 020235A}

Simply Blue SafeStain {Invitrogen, Cat#LC6060}

Sodium Phosphate {Sigma, Cat # S7907}

Millipore filters, 0.22 [symbol] m (Cat# SCGP U11 RE)

Trizma base {Sigma, Cat # T1503}

Resins:

Protein A Sepharose 4 Fast Flow {Amersham Pharmacia, Cat # 17097401}

(1st)

(2nd) Buffers:

Buffer A: 100 mM Tris HCl, pH 7.5
Buffer B: 100 mM Citrate-Phosphate, pH 3.5
Buffer C: 2M Tris
Buffer D: 1× PBS

Total Protein Extraction:

Medium containing expressed protein and cells was centrifuged in 1500×g/10 min/4° C. using SLA-3000 Rotor, and the Supernatant was filtrated using 0.22 μm filter. Aliquots were stored at −70° C. To concentrate the samples the aliquots were defrosted in a water bath at RT, and then combined into a single vial. Then the sample was concentred by ten-fold, by using Vivaflow 10 kDa device using PES membrane, to the final volume of 100-150 ml. The device was washed with 30 ml medium from residual protein, and the wash was then added to the sample. Sample was stored at 4° C., until the purification step.

Purification:

Affinity Column—Protein A Sepharose

The pH of the protein sample was elevated to 7.0, using 1M Tris, pH7.5, and the sample was filtered using a 0.22 μm filter (approximately 5% of the final volume).

A 1 ml Protein A 5/5 Column, previously equilibrated with buffers B and A listed above, was loaded with the protein sample at 1 ml/min. The column was washed with buffer A—up to 80CV—until O.D280 nm was less then 0.01, followed by elution with buffer B. The pH of the eluted fractions was elevated with 1/10 volume of buffer C that was placed in the empty tubes before the elution step. The eluted fractions were subjected to SDS-PAGE, followed by Coumassie staining. Finally, the eluted fractions containing the protein were subjected to dialysis with 2×2 L buffer D.

Bradford Quantization of the Purified Protein.

Bradford quantization of the purified protein was carried out using cold Bradford reagent, diluted 1:5 in ddH2O. BSA Standard commercial solution was made in the concentration range of 0.1 to 0.5 mg/ml. 200 μl of the diluted reagent and 10 μl of the standard/sample were added to microwell plates, in duplicates. The protein concentration was determined by comparison of the samples' O.D to the O.D of the known concentration of BSA.

Storage

The purified protein was stored in 1× PBS at −70° C.

### Example 5

Recognition of the CD40-Skipping 5 Protein by Anti-CD40 Antibody

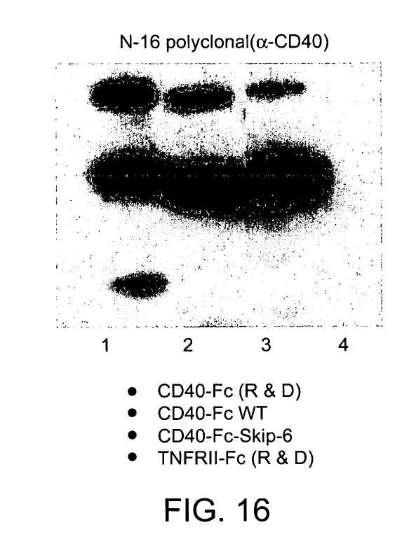

The CD40-skipping 5 protein was relatively unchanged by the purification, since it was easily recognised by a commercially available polyclonal antibody N-16, (polyclonal rabbit antibody from Santa Cruz (Cat num. Sc-974), which recognises the CD40 receptor, as can be seen from FIG. 16. FIG. 16 presents the results of Western blot analysis of the purified proteins as follows: line 1 presents CD-40wtEC-Fc protein, line 2 presents CD-40wt-Fc protein, line 3 presents CD-40 skipping6-Fc protein, and line 4 presents the TNFRII-Fc negative control. SDS-PAGE was performed as follows. The purified proteins were re-suspended in 30 μl 1× SDS-sample buffer containing 50 mM DTT (crude preparation). Following warming for 10 min and subsequent centrifugation, samples were loaded on Nu-PAGE gel buffer system (In-Vitrogen).

Following electrophoresis, for performing Western blots, gels were washed with cold transfer buffer for 15 min and taken for transfer to Nitrocellulose membranes for 60 min at 30 V using In-Vitrogen's transfer buffer and X-Cell II blot module. Following transfer, blots were blocked with TBS-5% skim milk (0.3% protein, 0.04% Tween-20) for at least 60 min. at room temperature or overnight at 4° C. Following blocking, blots were incubated with a commercially available N-16 antibody at 1 μg/ml for 1-3 hrs, washed with 0.05% Tween-20 in TBS, incubated with respective peroxidase-conjugated antibodies, washed with TBS-Tween-20 solution, followed by ECL. The results are shown in FIG. 16, demonstrating specific recognition between the N-16 antibody and the various purified CD-40 proteins.

### Example 6

FACS Analysis of sCD40 Binding to Mouse Fibroblasts Expressing Human CD154

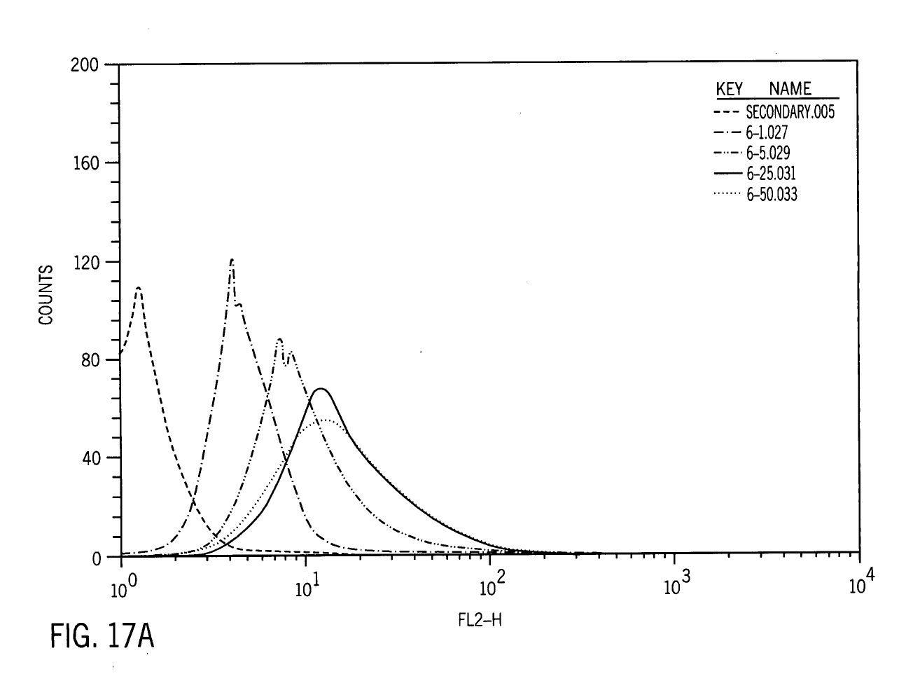

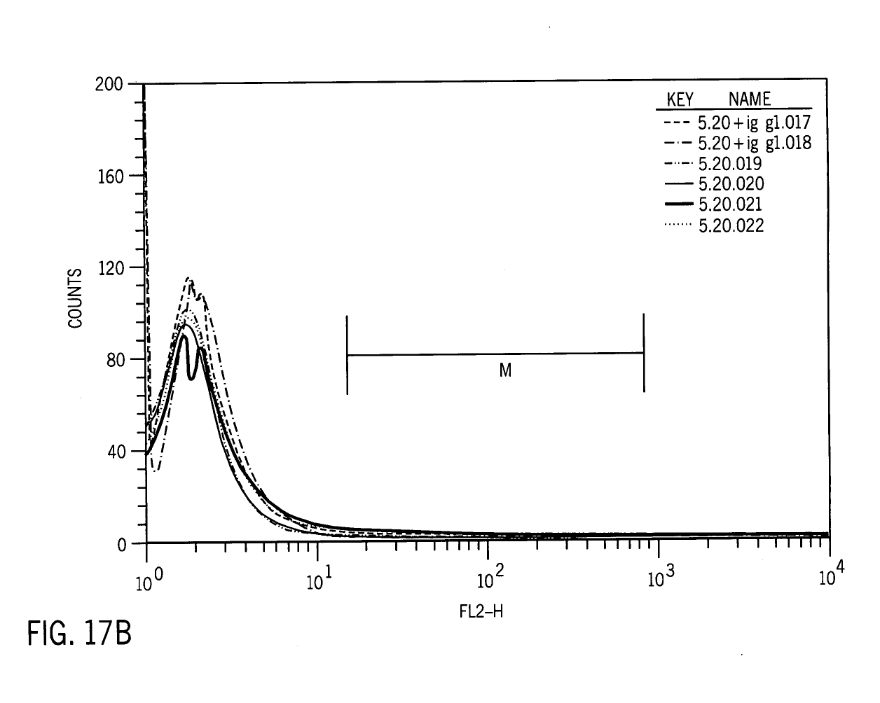

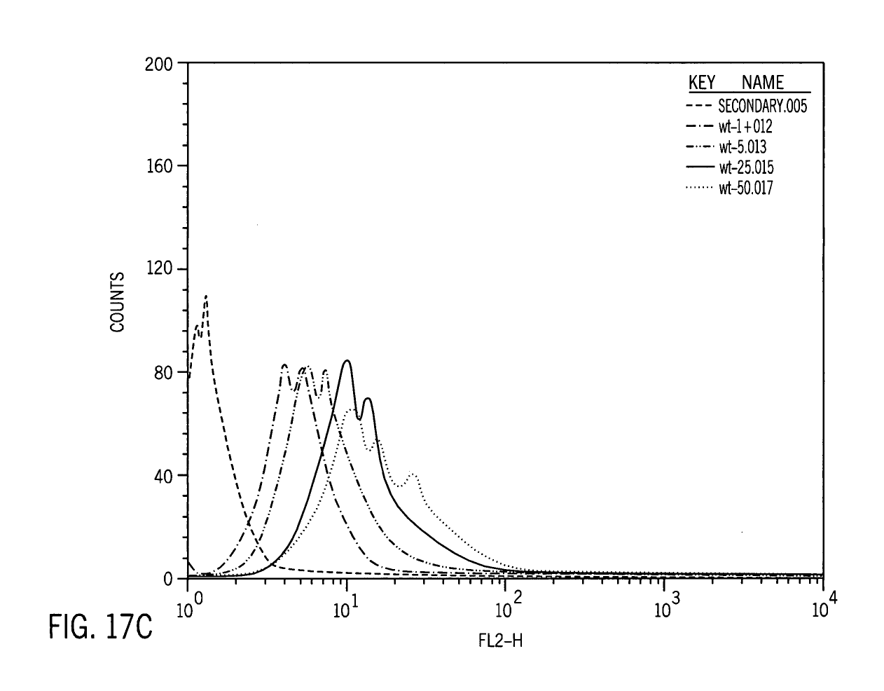

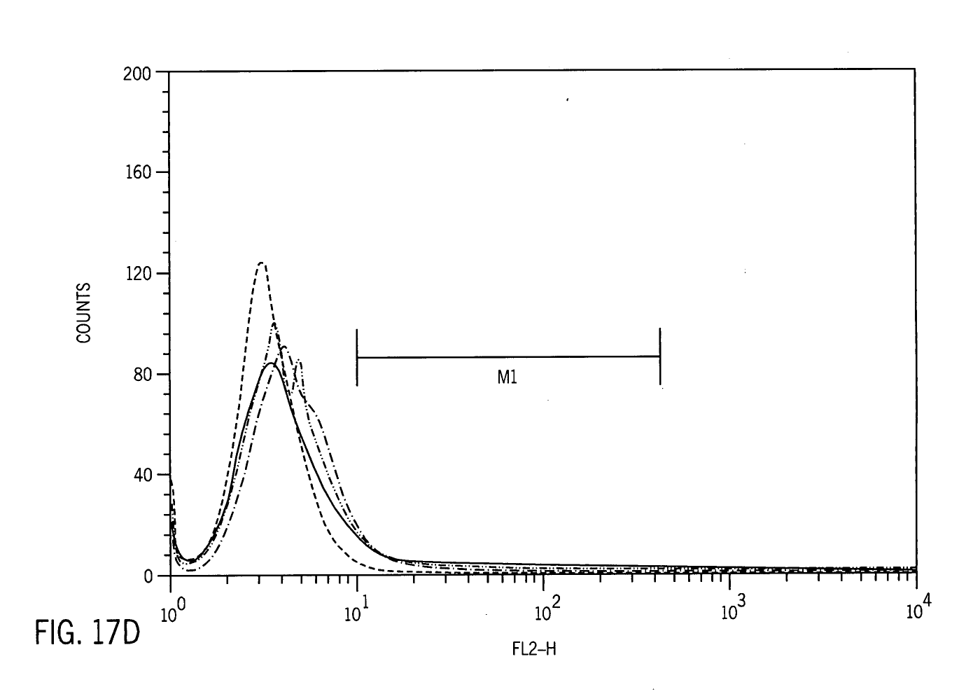

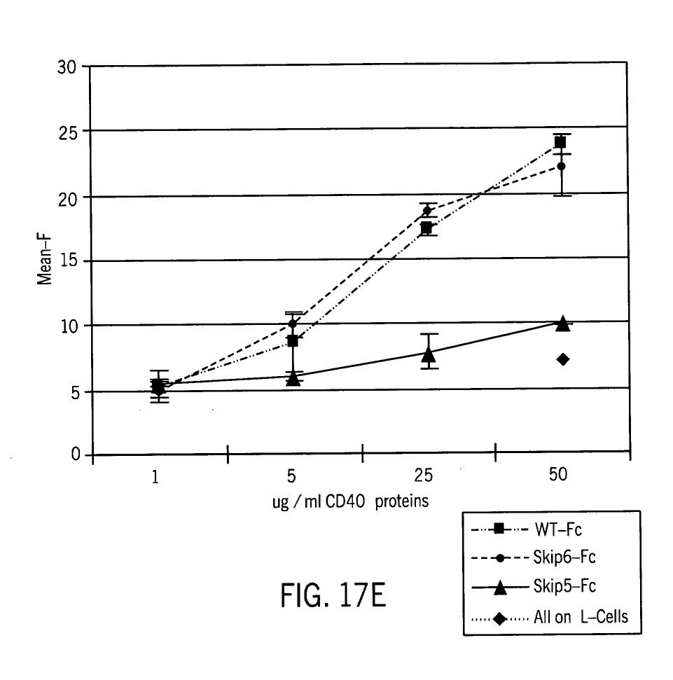

10⁶ mouse fibroblasts (stably transfected with full length human CD154) were incubated with either the known CD40 soluble variant, sCD40 WT (panel C, FIG. 17), the variant CD40-skipping 6 (panel A, FIG. 17) or the variant CD40-skipping 5 (panel B, FIG. 17). All CD40 variants were Fc tagged, and present at a concentration of 1-50 μg/ml at 4° C. for 60 min in total volume of 100 μl (0.5×106 cells). As a control, mouse fibroblast which do not express CD154 were used (panel D, FIG. 17). sCD40 binding was detected using PE-conjugated anti CD40 non-blocking antibody (clone EA-5). Analysis was performed using BD FACsCalibur. Panel E in FIG. 17 summarizes the mean fluorescence shift, as plotted versus various concentrations of CD40 protein.

Briefly, the FACS protocol was performed as follows. Mouse fibroblasts were trypsinized, and washed twice in PBS; cells were centrifuged at 1500 rpm for 10 min between washes. Next, the cells were re-suspended in FACS buffer (0.2% BSA and 0.02% sodium azide diluted 1/10 in PBS) to give 5-10×10⁶ cells mL. Cells were placed in FACS test-tubes at a volume of 100 or 200 microliters per tube.

Next, CD40 proteins were added (at concentrations of [1-50 μg/ml]), optionally with other treatments or controls, to the tubes containing the mouse fibroblast cells. The tubes were vortexed to mix and incubated for 1 hr at 4° C. in the dark.

5 ml PBS was added to each tube, after which the cells were pelleted to wash. The PBS buffer was removed by vacuum aspiration to reduce the volume back to 100 mL.

Next, 2 μL (1:50) anti CD40 non-blocking antibody (EA-5 mouse IgG1 PE-conjugated, obtained from Calbiochem) or controls (same isotype PE conjugated) were added to the tubes, and incubated for 30 min at 4° C. The process of washing was then repeated with 5 mL PBS. Next, 0.5 mL PBS was added to tubes, which were read in a FACS machine (BD FACSCalibur).

Alternatively after the second washing process, it is possible to perform fixation by adding 1 ml 4% paraformaldhyde to the cells and performing FACS analysis several days after the experiment. After fixation and before FACs analysis, the washing process should be repeated.

For this experiment, the controls included performing parallel assays with: mouse fibroblasts not expressing hCD154; non-relevant Fc (EA5, which is a mouse anti-CD40 antibody, see Malmborg Hager et al., Scandinavian J. 1 mm. 57:517-524 (2003) or non-Fc tagged proteins or purification mock; known CD40 soluble protein; secondary Ab only and isotype control only (isotype refers to an antibody control, featuring the same type of antibody but one which is not able to bind CD40). The axes are as follows: Y=cell counts; X=log scale of fluorescence intensity. M=A marker placed above the peak of positively stained cells on the histogram plot which provide the statistics of the stained population.

The FACS experiment showed significant binding of WT-sCD40-Fc and Skipping 6-sCD40-Fc to membrane CD154 while the skipping 5-sCD40-Fc exhibited different properties. This could be explained by different binding properties of this variant (see RANTES assay, Example 7 below)

### Example 7

Effect of the CD40 Variant on RANTES Secretion

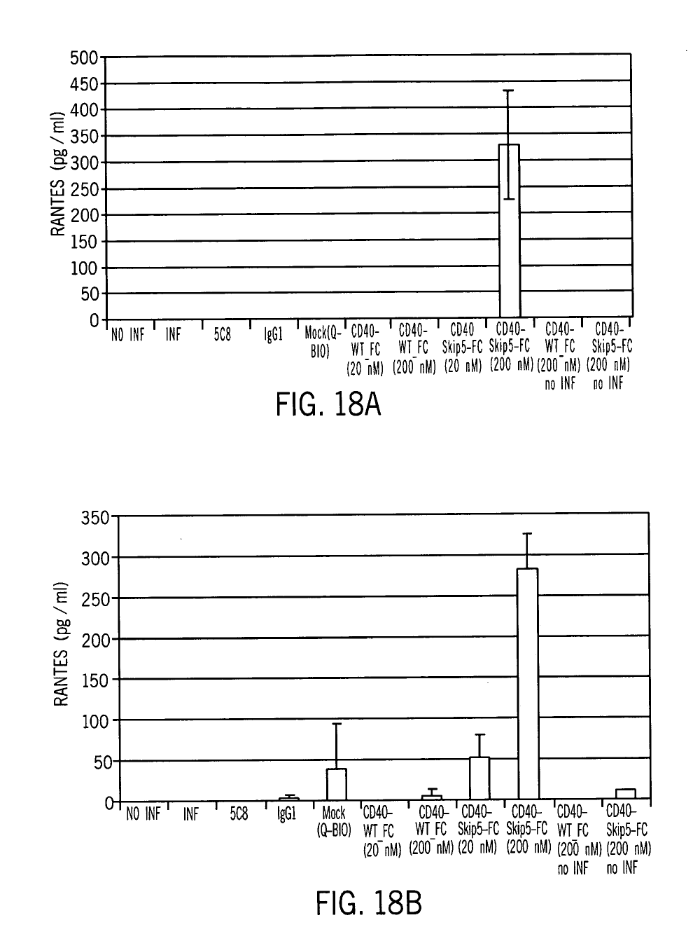

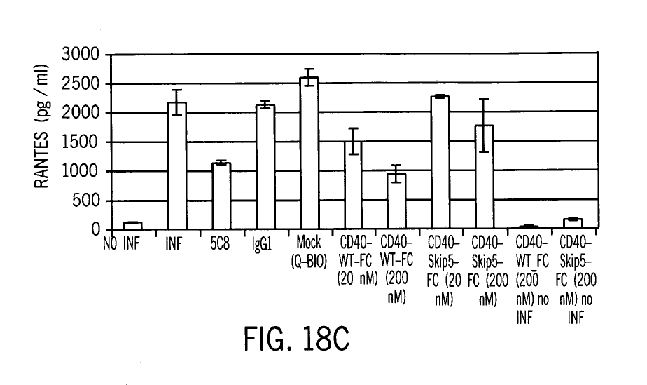

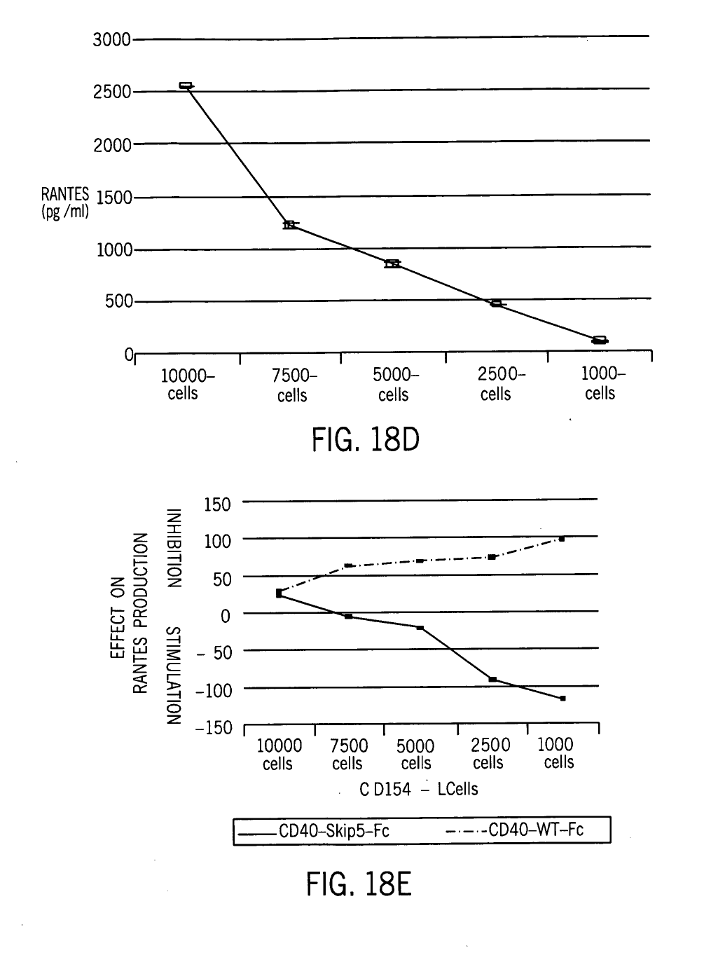

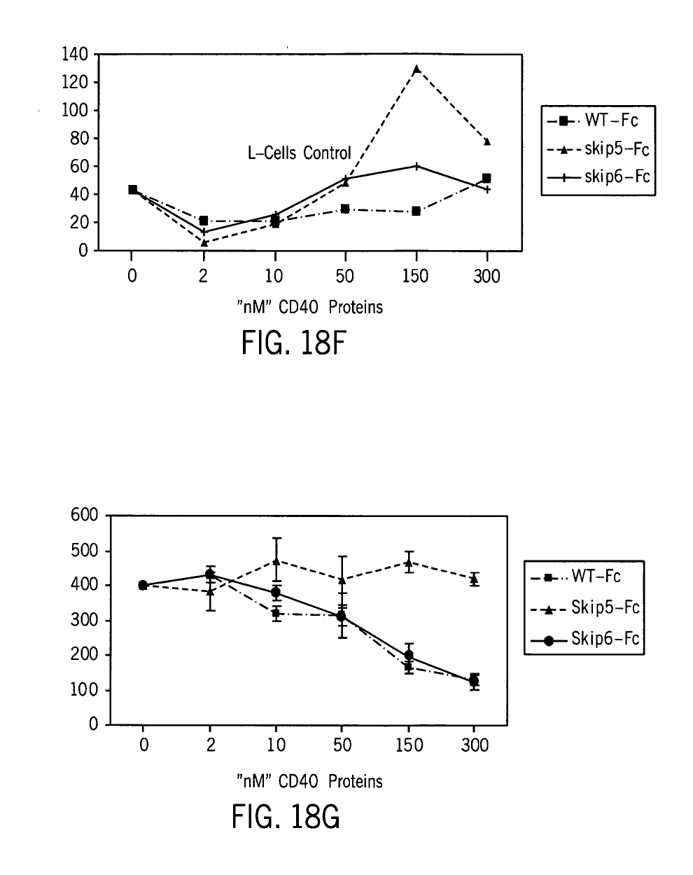

Skipping 5 protein was administered to a mixture of human peritoneal cells (HPMC cells), which express the CD40 receptor on their membrane, and mouse fibroblasts transfected to express the CD154 ligand. The ability of the soluble skipping 5 protein to compete with the CD40 membrane-bound receptor for binding to the CD154 ligand presented on the mouse fibroblasts was thus tested. This ability was measured by determining the resultant level of the cytokine RANTES, which is a cytokine indicative of T cell activation, as compared to when a positive control of interferon (which raises the level of RANTES via a CD40-related pathway) was administered alone. The results are shown in FIG. 18.

RANTES Cell Assay Protocol

HPMC cells were grown in M199+10% FCS (Biological Industries, Bet Ha'emek, Israel), trypsinized (using trypsine from Biological Industries, Bet Ha'emek, Israel, 5 ml/75 cm² cell culture flask), and recultured into 96-well plates. Cell should reach at least 80% confluence before further use.

Mouse fibroblasts/CD154-mouse fibroblasts were grown in DMEM+10% FCS (Biological Industries). Cells were trypsinized, pelleted (5 min 500 at rpm), counted (½ vol cells+½ vol trypan blue) and resuspended in M199+10% FCS. Cells were then diluted (10,000 or 5000 cells per ml).

CD40 proteins/antibodies were prepared in various concentrations in PBS according to the desired dose response/treatment (volume of 1 reaction should not exceed 20 microliters), followed by adding the same volume of PBS to the negative and positive controls.

The CD40 protein was added to 100 microliters of mouse fibroblasts or CD154-mouse fibroblasts in eppendorf tubes (final volume dependant on duplicates/triplicates) and incubated at R.T for 1 h at 200 rpm (rotation during incubation)

During this incubation time the HPMC were prepared, by removing medium and washing cells twice, and adding 100 microliters of fresh M199+10% FCS with or without 100 U/ml IFN (PeproTech, 50U/μl).

The CD40 L cells mixtures were overlaid on the HPMC (110 ul/well) and incubated O.N (at 37° C., 5% CO₂).

100 microliters were removed from supernatant and placed into new 96 well plates, followed by diluting the samples 1/10 in M199+10% FCS and using diluted samples for the ELISA RANTES test.

The cells in the experiment plate were checked for viability.

The initial assay, the results of which are demonstrated in FIGS. 18A and B, test control situations, in which there were only HPMC cells (FIG. 18A) or the mouse fibroblasts used in conjunction with the HPMC cells (FIG. 18B) were untransfected, and thus did not express CD154. There is therefore no ligand present in the system, and Rantes cannot be activated via the CD40-CD154 pathway, so its levels were relatively low, except for reactions where Skipping 5-sCD40-Fc was present and exhibited an agonistic effect which was Interferon dependent but CD154 independent.

FIG. 18C demonstrates the results of the experiment, in which the mouse fibroblasts used in conjunction with the HPMC cells were transfected and expressed CD154. Both ligand and receptor are present, therefore administration of the positive control interferon (INF) activated the CD40 pathway, and raised the Rantes level to 2000 pg/ml. Administration of an appropriate commercially available anti-CD40 antibody and WT-sCD40-Fc lowered this level to approximately 1000 pg/ml (50% inhibition), while Skipping 5-sCD40-Fc showed no inhibition in the complete system. The controls (hIgG1 and mock) did not influence any of the systems used.

As can be seen in FIGS. 18A-C, significant RANTES secretion is dependent on the presence of INF and CD154 presented on the mouse fibroblast. WT-SCD40-Fc and the CD154 neutralizing commercially available CD40 antibody exhibited inhibitory properties, while the controls (mock and IgG1) showed no influence on RANTES secretion. Interestingly the Skipping 5-sCD40-Fc variants exhibited agonistic properties as seen in FIGS. 18A and B in the absence of CD154. This phenomenon is masked in the complete system (FIG. 18C).

FIG. 18D demonstrates the results of RANTES inhibition/activation by sCD40 variants as dependency on the number of CD154+ mouse fibroblasts. As shown in FIG. 18D, the level of RANTES secretion was dependent on the number of CD154+ mouse fibroblasts. The same experiment was performed with a fixed concentration (200 nM) of sCD40 proteins. As shown in FIG. 18E, WT-CD40-Fc, shown in pink, exhibited increased inhibition (˜40-100%), while CD40 Skipping 5-Fc, shown in blue, exhibited a stimulatory effect on RANTES secretion.

FIG. 18F shows the results of a control experiment, in which the mouse fibroblast cells were untransfected and did not express CD154 (CD40 ligand). Therefore, administration of the various CD40 proteins had little influence on the level of RANTES.

FIG. 18G demonstrates the results of the dose response assay of inhibition of RANTES secretion by soluble CD40 proteins. In this experiment, the CD154 ligand is present (high cell number 10,000). The WT sCD40-Fc (shown in pink) and CD40 Skipping 6-Fc (shown in light blue) proteins exhibited an inhibitory effect and reduced the RANTES secretion in a dose dependent manner, while the CD40 skipping 5 protein (shown in yellow) did not cause any inhibition. The agonistic effect of CD40 skipping exon 5 variant was verified by several independent experiments.

### Example 8

mRNA Expression of the CD40 Variant

The following experiment was performed to determine the mRNA expression levels of endogenous CD40 skipping 5 variant, as compared to known CD40, using K562 erythroleukemia cell line.

Fishing of CD40 skipping 5 cDNA was done from RT-PCR of the K562 cell line. The K562 cell line was thawed and grown for 3 days in 40 ml. RNA was prepared from 30 ml cells. RNA concentration was 0.9 μg/ml. DNAse treatment was done using the Ambion kit. RT-PCT was done with oligo dt using 2 μg of superscript in 20 μlat 42C for 1 h, then 70C for 15 min.

PCR was done using 2 μl of this reaction. PCR conditions: annealing 62C/45 sec elongation/35 cycles. Four different fragments were purified from the 1.2% agarose gel and sequenced.

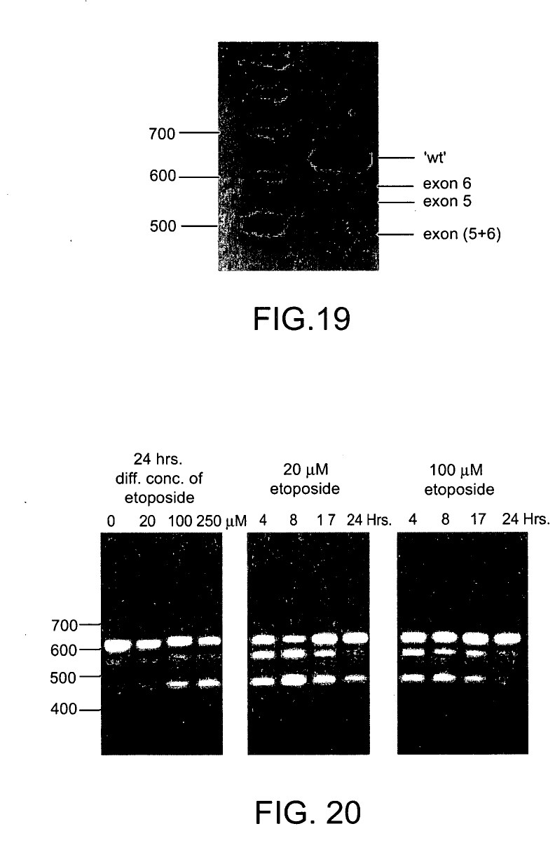

As demonstrated in FIG. 19, fragment of 550 bp was shown to be CD40 skipping 5 splice variants, while a fragment of 600 bp was found to be the CD40 skipping 6 variant, a fragment of 500 bp was found to be CD40 variant skipping both exons 5 and 6 and a fragment of 650 bp was found to be CD40 wild type.

The expression of different CD40 splice variants was detected in K562 erythroleukemia cell line, and is presented in FIG. 19. The secreted CD40 splice variant according to the present invention is “skipping exon 5 variant”, and is addressed in FIG. 19 as “exon 5”, while “wt” represents the original “wild type” form of the known CD40 molecule. The “exon (5+6)” is a membrane form of CD40 splice variant, described previously(Tone, M., et al., 2001, PNAS 98:1751-1756).

### Example 9

Functional Analysis of the CD40 Splice Variants in K562 Cells:

It is well accepted that CD40 mediates antiapoptotic and proliferative signaling for normal resting B cells (Tsubata, T, et al, 1993, Nature 384: 645-648). In contrast CD40 ligation in carcinoma cell lines results in growth inhibition and sensitizes these cells to apoptosis induced by a variety of agents, including TNF-, anti-Fas, and cytotoxic drugs (Eliopoulos, A. G., Oncogene 13:2243-2254). Furthermore, when exogenously expressed, CD40 has been shown to transduce apoptotic signals in certain cell lines of epithelial or mesenchymal origin. Due to the involvement of CD40 in apoptosis we attempted to test whether the expression pattern of the different variants of CD40 is altered as a response to apoptosis in K562 cells, and whether the expression pattern of skipping 5 differs from that of the known CD40.

Samples of RT reactions of K562 treated with etoposide were prepared and used for PCR using CD40 primers (35 cycles). Etoposide (Sigma) is known as double-stranded DNA breakage and apoptosis inducing agent. The RT reactions were checked before the analysis to exclude possible genomic contamination and to ensure similar cDNA concentrations in the different samples, using quantitation with GAPDH (not shown).

The results are shown in FIG. 20.

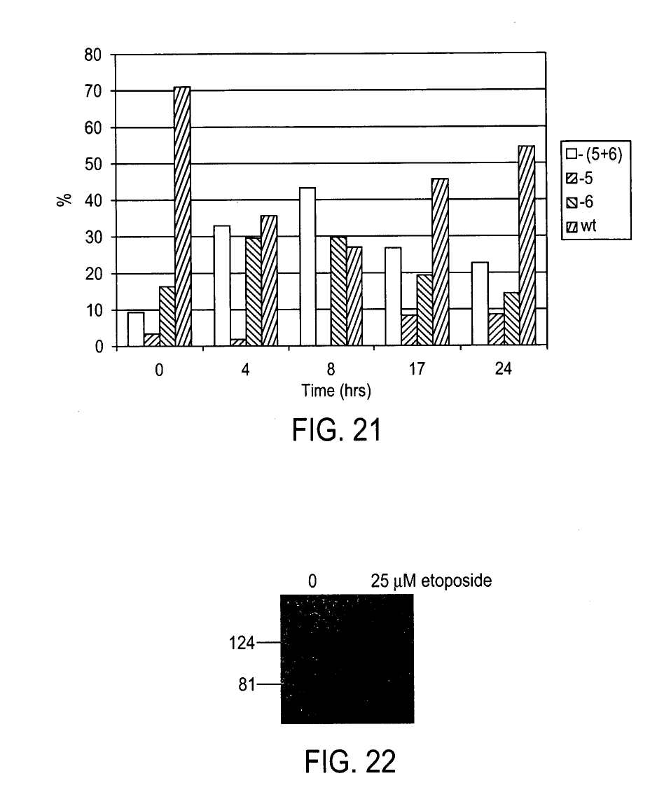

The bands resulting from the RT PCR reactions, demonstrated in FIG. 20, above were quantitated, and FIG. 21 presents the percentage of the expression of each splice form out of the total CD40 expression levels is presented for K562 cells treated with 20 μM Etoposide for various time intervals.

As can be seen from the results, while the known “wild type” CD40 molecule expression levels decrease upon double-stranded DNA breakage induced by Etoposide, the expression levels of the secreted CD40 splice variant skipping exon 5 (“skipping 5”) increase. The optimal effect is observed at 17 hours of treatment of K562 cells with 20 uM Etoposide. The skipping 5 mRNA transcript therefore has a physiological expression pattern which is different from that of the known CD40 receptor protein, when apoptosis is induced in erythroleukemic cells. The skipping 5 protein is thus novel over the known CD40 protein, having a novel cellular expression pattern, in addition to its possessing an amino acid sequence which is distinct from that of the known CD40 proteins (skipping 5 contains a unique tail not present in the known CD40 molecules).

Since K562 cells do not have active p53, these cells were tested to determine whether they still enter apoptosis after treatment with etoposide in the relevant time frame. For this purpose K562 cells were treated with 25 μM etoposide for 17 hrs, and the activation of caspases, one of the hallmarks of apoptosis, was measured. To measure caspase activation, the cells were lysed, immunoblotted and PARP, a known substrate for caspase-3, was probed using anti cleaved PARP antibody (Cell Signaling, Beverly, Mass.). The results are shown in FIG. 22, and they demonstrate the appearance of a band in the expected size in the treated cells, thus indicating that the effect seen by etoposide may be part of the apoptotic machinery.

The effect on the expression of the secreted splice form compared to the expression of the known CD40 in the course of apoptosis can be explained by the dominant negative nature of the secreted form. While the original antiapoptotic CD40 is repressed during DNA damage and subsequent apoptosis, the CD 40 skipping 5 secreted form might compete with the known CD40 form by binding to the ligand without subsequent signaling. Therefore, the secreted molecule, which is overexpressed upon DNA damage, might act as an antagonist of the original CD40 molecule and can be utilized accordingly, to disrupt the CD40 receptor-ligand interaction.

### Example 10

Treatment of Atherosclerosis by Administering CD40-Skipping 5 Splice Variant Protein

A human subject diagnosed with atherosclerosis is treated with a CD40-skipping 5 splice variant protein to reduce the symptoms associated with the disease. A CD40-skipping 5 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. Additional doses are administered as warranted from about daily to about weekly.

### Example 11

Treatment of Cancer by Administering CD40-Skipping 5 Splice Variant Protein

A subject diagnosed with colorectal cancer is treated with a CD40-skipping 5 splice variant protein to reduce the symptoms associated with the disease. A CD40-skipping 5 splice variant protein is suspended in a suitable buffer for subcutaneous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms and response of the cancer to treatment. Depending on the physical characteristics, additional doses are monitored from about daily to about weekly.

### Example 12

Treatment of a Chronic Inflammatory Disease by Administering CD40-Skipping 5 Splice Variant Protein

A subject diagnosed with inflammatory bowel syndrome is treated with a CD40-skipping 5 splice variant protein to reduce the symptoms associated with the disease. A CD40-skipping 5 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms. Depending on the physical characteristics, additional doses are monitored from about daily to about weekly.

### Example 13

Treatment of Atherosclerosis by Gene Therapy with CD40-Skipping 5 Splice Variant

A subject diagnosed with atherosclerosis is treated by administering a gene therapy construct capable of expressing a CD40-skipping 5 splice variant protein to reduce the symptoms associated with the disease. The CD40-skipping 5 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

### Example 14

Treatment of Cancer by Gene Therapy with CD40-Skipping 5 Splice Variant

A subject diagnosed with cancer is treated by administering a gene therapy construct capable of expressing a CD40-skipping 5 splice variant protein to reduce the symptoms associated with the disease. The CD40-skipping 5 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

### Example 15

Treatment of Chronic Inflammatory Disease by Gene Therapy with CD40-Skipping 5 Splice Variant

A subject diagnosed with chronic inflammatory disease is treated by administering a gene therapy construct capable of expressing a CD40-skipping 5 splice variant protein to reduce the symptoms associated with the disease. The CD40-skipping 5 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

The descriptions given are intended to exemplify, but not limit, the scope of the invention. Additional embodiments are within the claims.

What is claimed is:

1. A method for treating a disease in which it is desired to increase the activity of the immune system in a subject, the method comprising administering to said subject a therapeutically effective amount of a CD40 skipping 5 protein, wherein said CD40 protein is selected from the group consisting of
i) a polypeptide comprising the amino acid sequence depicted in SEQ ID No: 1;
ii) an isolated chimeric polypeptide encoding for CD40 skipping 5, comprising a first amino acid sequence being at least about 90% homologous to amino acids 1-135 corresponding to the known CD40 sequence SEQ ID NO: 3 and a second amino acid sequence being at least about 70% homologous to a polypeptide having the sequence VRPKTWLCNRQAQTRLMLSVVPRIG, wherein said first and said second amino acid sequences are contiguous and in a sequential order;
iii) an isolated chimeric polypeptide encoding for a tail of CD40 skipping 5, comprising a polypeptide having the sequence VRPKTWLCNRQAQTRLMLSVVPRIG; and
iv) an isolated chimeric polypeptide encoding for an edge portion of CD40 skipping 5 corresponding to SEQ ID NO: 1, comprising a polypeptide having a length “n”, wherein n is at least about 10 amino acids in length, wherein at least two amino acids comprise AV having a structure as follows (numbering according to SEQ ID NO:1): a sequence starting from any of amino acid numbers 135−x to 135 and ending at any of amino acid numbers 136+((n−2)−x), in which x varies from 0 to n−2, such that the value ((n−2)−x) is not allowed to be larger than 24.

2. The method of claim 1, wherein said disease is a hematological malignancy or cancer.

3. The method of claim 2, wherein said hematological malignancy or cancer is selected from the group consisting of leukemia, lymphoma, multiple myeloma, epithelial neoplasia, nasopharyngeal carcinoma, osteosarcoma, neuroblastoma bladder carcinoma, ovary carcinoma, liver carcinoma, breast cancer, colorectal cancer, and AIDS-related lymphoma.

4. The method of claim 1, wherein said disease is associated with bone loss.

5. The method of claim 4 wherein said disease is selected from the group consisint of osteoporosis, osteonecrosis and inflammatory arthritis.

6. The method of claim 5, wherein said disease is an autoimmune disease.

7. The method of claim 6, wherein said autoimmune disease is selected from the group consisting of lupus nephritis, systemic lupus erythematosus, rheumatoid arthritis, multiple sclerosis, inflammatory bowel disease (IBD), ulcerative colitis, Crohn's disease, hematological malignancies, Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Lupus (SLE), Grave's disease, myasthenia gravis, autoimmune hemolytic anemia, autoimmune thrombocytopenia, and asthma.

9. The method of claim 1, wherein said disease is impaired renal function.

10. The method of claim 9, wherien impaired renal function is due to chronic renal failure, haemodialysis or chronic ambulatory peritoneal dialysis (CAPD).

11. The method of claim 1, wherein said subject is a human.

12. The method of claim 1, wherein said subject has a weakened immune system.

13. The method of claim 1, wherein said CD40 protein comprises the 5 amino acid sequence depicted in SEQ ID NO:1.

14. The method of claim 1, wherein said CD40 protein is an isolated chimeric polypeptide encoding for CD40 skipping 5, comprising a first amino acid sequence being at least about 90% homologous to amino acids 1-135 corresponding to the known CD40 sequence SEQ ID NO:3 and a second amino acid sequence being at least about 70% homologous to a polypeptide having the sequence VRPKTWLCNRQAQTRLMLSVVPRIG, wherein said first and said second amino acid sequences are contiguous and in a sequential order.

15. The method of claim 1, wherein said CD40 protein is an isolated chimeric polypeptide encoding for a tail of CD40 skipping 5, comprising a polypeptide having the sequence VRPKTWLCNRQAQTRLMLSVVPRIG.

16. The method of claim 1, wherein said CD40 protein is an isolated chimeric polypeptide encoding for an edge portion of CD40 skipping 5 corresponding to SEQ ID NO:1, comprising a polypeptide having a length “n”, wherein n is at least about 10 amino acids in length wherein at least two amino acids comprise AV having a structure as follows (numbering according to SEQ ID NO:1): a sequence starting from any of amino acid numbers 135−x to 135 and ending at any of amino acid numbers 136+((n−2)−x), in which x varies from 0 to n−2, such that the value ((n−2)−x) is not allowed to be larger than 24.


SEQUENCE LISTING (30 sequences)

SEQ ID NO: 1 - protein, 160 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIAVRPKTWLCNRQAQTRLMLSVVPRIG

SEQ ID NO: 2 - DNA, 526 bp, Homo sapiens
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctgtgagaccaaagacctggttgtgcaacaggcaggcacaaacaagactgatgttgtctgtggtccccaggatcggctgagagccctggtggtgatccccatcatcttcgggatcctgtttgc

SEQ ID NO: 3 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 4 - DNA, 910 bp, Homo sapiens
gcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacg

SEQ ID NO: 5 - protein, 25 aa, Homo sapiens
VRPKTWLCNRQAQTRLMLSVVPRIG

SEQ ID NO: 6 - protein, 18 aa, Homo sapiens
RPKTWLCNRQAQTRLMLS

SEQ ID NO: 7 - DNA, 32 bp, Artificial Sequence
  40wt5′ oligonucleotide primer
actagatatcatggttcgtctgcctctgcagt

SEQ ID NO: 8 - DNA, 30 bp, Artificial Sequence
  40wt3′ oligonucleotide primer
aagcagatcttatctcagccgatcctgggg

SEQ ID NO: 9 - DNA, 792 bp, Homo sapiens
tttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagataagatctgggtaaacgcagttccaagtaaatgaatcgtttttaaaataacaaatcaattgttttataatat

SEQ ID NO: 10 - DNA, 5234 bp, Artificial Sequence
  oligonucleotide vector pTen21
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatccaagcttaagagctcgagcggccgcctgcagccggtacccagatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 11 - DNA, 21 bp, Artificial Sequence
  OQBT51 oligonucleotide primer
gcatttgaggatgccgggacc

SEQ ID NO: 12 - DNA, 20 bp, Artificial Sequence
  OQBT31 oligonucleotide primer
cataatcaaagaatcgtacg

SEQ ID NO: 13 - DNA, 5909 bp, Artificial Sequence
  Oligonucleotide Vector pTen21-Fc
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatccaagcttaagagctcgagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 14 - DNA, 684 bp, Homo sapiens
acacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctgtgagaccaaagacctggttgtgcaacaggcaggcacaaacaagactgatgttgtctgtggtccccaggatcggctaagcttggatctgggtaaacgcagttccaagtaaatgaatcgtttttaaaataacaaatcaattgttttataatat

SEQ ID NO: 15 - DNA, 914 bp, Homo sapiens
aacccaacacaatatattatagttaaataagaattattatcaaatcatttgtatattaattaaaatactatactgtaaattacatttttatttacaatcagctgaaagcctaacgcagatcggatccgatatccaagcttaagagctcgagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttata

SEQ ID NO: 16 - DNA, 40 bp, Artificial Sequence
  40Fc3′ oligonucleotide primer
cacaagatctgggctctacgtatctcagccgatcctgggg

SEQ ID NO: 17 - DNA, 35 bp, Artificial Sequence
  4053′ oligonucleotide primer
gtaaggatccaagcttagccgatcctggggaccac

SEQ ID NO: 18 - DNA, 6484 bp, Artificial Sequence
  Oligonucleotide vector pTen21-CD40wtEC-Fc
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagatacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 19 - DNA, 1477 bp, Homo sapiens
ccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagatacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaat

SEQ ID NO: 20 - DNA, 39 bp, Artificial Sequence
  5Fc3′ oligonucleotide primer
cacaagatctgggctctacgtagccgatcctggggacca

SEQ ID NO: 21 - DNA, 6385 bp, Artificial Sequence
  pTen21-CD40_Skipping 5-Fc oligonucleotide vector
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctgtgagaccaaagacctggttgtgcaacaggcaggcacaaacaagactgatgttgtctgtggtccccaggatcggctacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 22 - DNA, 1374 bp, Homo sapiens
tatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctgtgagaccaaagacctggttgtgcaacaggcaggcacaaacaagactgatgttgtctgtggtccccaggatcggctacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttataa

SEQ ID NO: 23 - DNA, 20 bp, Artificial Sequence
  Stu40 oligonucleotide primer
caccatctgcacctgtgaag

SEQ ID NO: 24 - protein, 203 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTRSPGSAESPGGDPHHLRDPVCHPLGAGLYQKGGQEANQ

SEQ ID NO: 25 - protein, 244 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGESWTMGPGESLGRWELKGEMRHTGTLDGKKGRGGSLGVWYHSSATYLGSLGKSLPLS

SEQ ID NO: 26 - protein, 191 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGLGLE

SEQ ID NO: 27 - protein, 237 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGESWTMGPGESLGRSPGSAESPGGDPHHLRDPVCHPLGAGLYQKGGQEANQ

SEQ ID NO: 28 - protein, 166 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIAVRPKTWLCNRQAQTRLMLSVVSPGQWALEKA

SEQ ID NO: 29 - protein, 151 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIDICQPHFPKDRGLNLLM

SEQ ID NO: 30 - protein, 144 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICE